SEQUENCE LISTING (7 sequences)

SEQ ID NO: 1 - DNA, 58 bp, Artificial sequence
  Synthetic
ccgggctgacatgatctttacttatctcgagataagtaaagatcatgtcagctttttg

SEQ ID NO: 2 - DNA, 58 bp, Artificial sequence
  Synthetic
aattcaaaaagctgacatgatctttacttatctcgagataagtaaagatcatgtcagc

SEQ ID NO: 3 - DNA, 58 bp, Artificial sequence
  Synthetic
ccggcgatgtatggaaactgctatactcgagtatagcagtttccatacatcgtttttg

SEQ ID NO: 4 - DNA, 58 bp, Artificial sequence
  Synthetic
aattcaaaaacgatgtatggaaactgctatactcgagtatagcagtttccatacatcg

SEQ ID NO: 5 - DNA, 58 bp, Artificial sequence
  Synthetic
ccgggtggccattctgctctatcttctcgagaagatagagcagaatggccactttttg

SEQ ID NO: 6 - DNA, 58 bp, Artificial sequence
  Synthetic
aattcaaaaagtggccattctgctctatcttctcgagaagatagagcagaatggccac

SEQ ID NO: 7 - protein, 6 aa, Artificial sequence
  Synthetic
RRWWCR

(19) United States
(12) Patent Application Publication
(10) Pub. No.: US 2017/0044255 A1
(43) Pub. Date: Feb. 16, 2017

(54) REDUCING CUTANEOUS SCAR FORMATION AND TREATING SKIN CONDITIONS

(71) Applicant: Northwestern University, Evanston, IL (US)

(72) Inventors: Wei Xu, Chicago, IL (US); Seok Jong Hong, Northbrook, IL (US); Robert D. Galiano, Chicago, IL (US); Thomas A. Mustoe, Evanston, IL (US)

(21) Appl. No.: 15/241,841

(22) Filed: Aug. 19, 2016

Related U.S. Application Data
(63) Continuation of application No. 14/469,823, filed on Aug. 27, 2014, now abandoned.
(60) Provisional application No. 61/870,607, filed on Aug. 27, 2013.

(51) Int. Cl. C07K 16/28; C12N 15/113

(52) U.S. Cl. CPC C07K 16/28; C12N 15/1138; C07K 2317/76; C12N 2310/14; C12N 2310/141; C12N 2320/31

(57) ABSTRACT

The present invention provides methods of reducing cutaneous scar formation by treating a cutaneous wound with a composition comprising a therapeutic agent that is a sodium channel blocker and/or an inhibitor of the Naₓ/SCN7A pathway. The present invention also provides wound cover components impregnated with such compositions, kits composed of such compositions with a wound dressing or sterile wipe, and mixtures of such compositions with a topical component (e.g., cream, ointment, or gel) suitable for application to a cutaneous wound. The present invention also provides compositions, kits, devices, and methods for treating skin conditions (e.g., dermatitis, psoriasis, or other skin conditions) with such compositions and devices. Examples of such therapeutic agents include, but are not limited to, an inhibitor of a gene or protein selected from: ENac, COX-2, PGE2, PI3K, PKB, Naₓ Prss8, IL-1β, IL-8, SAPK, Erk gene, p38 gene, PAR2, S100A8, S100A9, S100A12.

The present application is a continuation of U.S. patent application Ser. No. 14/469,823, filed Aug. 27, 2014, which claims priority to U.S. Provisional Application Ser. No. 61/870,607, filed Aug. 27, 2013, each of which is herein incorporated by reference in its entirety.

### FIELD OF THE INVENTION

The present invention provides methods of reducing cutaneous scar formation by treating a cutaneous wound with a composition comprising a therapeutic agent that is a sodium channel blocker and/or an inhibitor of the Naₓ/SCN7A pathway. The present invention also provides wound cover components impregnated with such compositions, kits composed of such compositions with a wound dressing or sterile wipe, and mixtures of such compositions with a topical component (e.g., cream, ointment, or gel) suitable for application to a cutaneous wound. The present invention also provides compositions, kits, devices, and methods for treating skin conditions (e.g., dermatitis, psoriasis, or other skin conditions) with such compositions and devices. Examples of such therapeutic agents include, but are not limited to, an inhibitor of a gene or protein selected from: ENac, COX-2, PGE2, PI3K, PKB, Naₓ Prss8, IL-1β, IL-8, SAPK, Erk gene, p38 gene, PAR2, S100A8, S100A9, S100A12.

### BACKGROUND

The repair of injured skin tissue is a fundamental biological process essential to the continuity of life, but with potential for dysregulation and overcompensation. Derangements in healing can lead to excessive (hypertrophic) scar formation, for which there are a paucity of therapeutic options (1-4). Injury to the epidermis results in loss of epithelial barrier function which is not restored until the lipid barrier (stratum corneum) becomes fully competent. Of relevance to scarring, it has been demonstrated that scars have a perturbed barrier function as compared to unwounded skin for up to one year post-injury (5).

There is an extensive body of literature implicating the importance of the epidermal barrier function in cutaneous homeostasis (6, 7). Specifically, epithelial dehydration results in compensatory changes in the injured skin including up-regulation of inflammatory cytokines and activation of fibroblasts, which are implicated in hypertrophic scarring. Furthermore, it is notable that many skin disorders such as atopic dermatitis, ichthyosis and psoriasis have an impaired barrier function, and various emollients, and moisturizers improve their symptoms and reduce dermal inflammation by improving barrier function (8).

### SUMMARY OF THE INVENTION

The present invention provides methods of reducing cutaneous scar formation by treating a cutaneous wound with a composition comprising a therapeutic agent that is a sodium channel blocker and/or an inhibitor of the Naₓ/SCN7A pathway. The present invention also provides wound cover components impregnated with such compositions, kits composed of such compositions with a wound dressing or sterile wipe, and mixtures of such compositions with a topical component (e.g., cream, ointment, or gel) suitable for application to a cutaneous wound. The present invention also provides compositions, kits, devices, and methods for treating skin conditions (e.g., dermatitis, psoriasis, or other skin conditions) with such compositions and devices. Examples of such therapeutic agents include, but are not limited to, an inhibitor of a gene or protein selected from: ENac, COX-2, PGE2, PI3K, PKB, Naₓ Prss8, IL-1β, IL-8, SAPK, Erk gene, p38 gene, PAR2, S100A8, S100A9, S100A12.

In certain embodiments, the present invention provides methods of reducing cutaneous scar formation comprising: applying a composition to a cutaneous wound of a subject, wherein the composition comprises a therapeutic amount of a therapeutic agent, wherein the therapeutic agent: i) is a sodium channel blocker, and/or ii) is an inhibitor of the Naₓ/SCN7A pathway.

In particular embodiments, the present invention provides compositions comprising: a) a therapeutic agent that is: i) is a sodium channel blocker, and/or ii) is an inhibitor of the Naₓ/SCN7A pathway; and b) a topical component suitable for application to a cutaneous wound, wherein the therapeutic agent is in the topical component.

In other embodiments, the present invention provides kits comprising: a) a composition comprising a therapeutic agent that is: i) is a sodium channel blocker, and/or ii) is an inhibitor of the Naₓ/SCN7A pathway; and b) a wound treatment component selected from the group consisting of: a sterile wipe and a wound dressing.

In some embodiments, the present invention provides a device for treating wounds comprising: a wound cover component configured to at least partially cover a cutaneous wound, wherein the wound cover component is impregnated with a therapeutic agent that is: i) is a sodium channel blocker, and/or ii) is an inhibitor of the Naₓ/SCN7A pathway.

In further embodiments, the present invention provides methods of treating dermatitis or psoriasis comprising: applying a composition to a dermatitis or psoriasis affected skin surface of a subject such that the symptoms of the dermatitis or psoriasis are reduced or eliminated, wherein the composition comprises a therapeutic amount of a therapeutic agent that is: i) is a sodium channel blocker, and/or ii) is an inhibitor of the Naₓ/SCN7A pathway.

In some embodiments, the therapeutic agent is an inhibitor of at least one of the following: a) epidermal sodium channel (ENac) mRNA or protein, b) cyclooxygenase-2 (COX-2) mRNA or protein, c) prostaglandin E2 (PGE2) mRNA or protein, d) phosphoinositide 3 kinase (PI3K) mRNA or protein, e) protein Kinase B (PKB or Akt) mRNA or protein, f) Naₓ (SCN7A) mRNA or protein, g) Prss8 mRNA or protein, h) interleukin-1β (IL-1β) mRNA or protein, i) interleukin 8 (IL-8) mRNA or protein, j) SAPK mRNA or protein, k) Erk mRNA or protein, l) p38 mRNA or protein, m) PAR2 mRNA or protein, n) S100A8 mRNA or protein, o) S100A9 mRNA or protein, and p) S100A12 mRNA or protein. In particular embodiments, the agent inhibits the gene of the recited protein. In other embodiments, the recited protein (or gene encoding the protein) is human.

In some embodiments, the present invention provides methods of reducing cutaneous scar formation comprising: applying a composition to a cutaneous wound of a subject (e.g., a mammalian subject, human subject, a dog, a cat, a horse, etc.), wherein the composition comprises a therapeutic amount of a therapeutic agent selected from the group consisting of: a) an epidermal sodium channel (ENac) inhibitor; b) a cyclooxygenase-2 (COX-2) inhibitor; c) a prostaglandin E2 (PGE2) inhibitor; d) a phosphoinositide 3 kinase (PI3K) inhibitor, and e) a protein Kinase B (PKB or Akt) inhibitor.

In certain embodiments, said treating is repeated on at least 3 separate days (e.g., at least 3, 4, 5, . . . 10 . . . 15 . . . 20 . . . or 30 separate days). In some embodiments, said treating is repeated daily or bi-daily for at least a week.

In particular embodiments, the present invention provides methods of treating a skin condition (e.g., such as dermatitis (e.g., eczema, rash, seborrheic dermatitis, etc.), psoriasis, etc.) comprising: applying a composition to a dermatitis skin surface of a subject (e.g., a mammalian subject, human subject, a dog, a cat, a horse, etc.) such that the symptoms of dermatitis are reduced or eliminated, wherein the composition comprises a therapeutic amount of a therapeutic agent selected from the group consisting of: a) an epidermal sodium channel (ENac) inhibitor; b) a cyclooxygenase-2 (COX-2) inhibitor; c) a prostaglandin E2 (PGE2) inhibitor; d) a phosphoinositide 3 kinase (PI3K) inhibitor; and e) a protein Kinase B (PKB or Akt) inhibitor.

In certain embodiments, the present invention provides methods of reducing cutaneous scar formation comprising: applying a composition to a cutaneous wound of a subject such that the resulting scar formed from healing of the wound is smaller and/or less visible than would be present if the wound were un-treated, wherein the composition comprises a therapeutic agent is selected from the group consisting of: a) an epidermal sodium channel (ENac) inhibitor; b) a cyclooxygenase-2 (COX-2) inhibitor; c) a prostaglandin E2 (PGE2) inhibitor; d) a phosphoinositide 3 kinase (PI3K) inhibitor; and e) a protein Kinase B (PKB or Akt) inhibitor. In certain embodiments, the resulting scar has a lower skin elevation index score than if the wound were un-treated. In other embodiments, the therapeutic amount is about 0.1 to 10 mg per 1 cm² of the cutaneous wound.

In particular embodiments, the present invention provides compositions comprising: a) a therapeutic agent selected from the group consisting of: i) an epidermal sodium channel (ENac) inhibitor; ii) a cyclooxygenase-2 (COX-2) inhibitor; iii) a prostaglandin E2 (PGE2) inhibitor; iv) a phosphoinositide 3 kinase (PI3K) inhibitor; and v) a protein Kinase B (PKB or Akt) inhibitor; and b) a topical component suitable for application to a cutaneous wound, wherein the therapeutic agent is in the topical component. In certain embodiments, the topical component is selected from the group consisting of: a topical cream, a topical foam, a topical gel, a topical lotion and a topical ointment. In other embodiments, the concentration of the therapeutic agent in the composition is about 0.1% to about 1%).

In some embodiments, the present invention provides kits comprising: a) a composition comprising a therapeutic agent selected from the group consisting of: i) an epidermal sodium channel (ENac) inhibitor; ii) a cyclooxygenase-2 (COX-2) inhibitor; iii) a prostaglandin E2 (PGE2) inhibitor; iv) a phosphoinositide 3 kinase (PI3K) inhibitor; and v) a protein Kinase B (PKB or Akt) inhibitor; and b) a wound treatment component selected from the group consisting of: a sterile wipe and a wound dressing. In particular embodiments, the wound dressing is selected from the group consisting of: gauze, adhesive bandage, films, gels, foams, hydrocolloids, alginates, hydrogels, polysaccharide pastes, granules and beads.

In further embodiments, the present invention provides devices for treating wounds comprising: a wound cover component configured to at least partially cover a cutaneous wound, wherein the wound cover component is impregnated with a therapeutic agent selected from the group consisting of: i) an epidermal sodium channel (ENac) inhibitor; ii) a cyclooxygenase-2 (COX-2) inhibitor; iii) a prostaglandin E2 (PGE2) inhibitor; iv) a phosphoinositide 3 kinase (PI3K) inhibitor; and v) a protein Kinase B (PKB or Akt) inhibitor.

In certain embodiments, the wound cover component is impregnated with the therapeutic agent such that the therapeutic agent migrates out of the wound cover component when it is applied to a cutaneous wound. In further embodiments, the wound cover component is selected from the group consisting of: gauze, an adhesive bandage, and a film.

In some embodiments, the ENac inhibitor is selected from the group consisting of: amiloride, triamterene, benzamil, GS9411, P-365, and pyrazine derivatives (see, e.g., U.S. Pat. No. 8,372,845, which is herein incorporated by reference for compounds recited therein). Additional ENac inhibitors can be found by using the screening methods of U.S. Pat. No. 8,105,792, herein incorporated by reference. In further embodiments, the COX-2 inhibitor is selected from the group consisting of: celecoxib (Celebrex), valdecoxib (Bextra), rofecoxib (Vioxx), diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, tolmetin, acetaminophen, and miRNA targeting the COX-2 gene (e.g., miR-26b, miR-199a*, miR-16, miR-101a, miR-143, miR-144, miR-145, miR-199a, miR-542-3p, and miR-543). In particular embodiments, the PGE2 inhibitor is selected from the group consisting of: curcumin, SC-560, AH6809, sulforaphane, wagonin, rifampin, and miRNA targeting the prostaglandin E2 gene. In other embodiments, the PI3K inhibitor is selected from the group consisting of: LY294002, Wortmannin, demethoxyviridin, Perifosine, CAL101, PX-866, IPI-145, BAY 80-6946, BEZ235, TGR 1202, SF1126, INK1117, GDC-0941, BKM120, XL147, XL765, Palomid 529, GSK1059615, ZSTK474, PWT33597, IC87114, TG100-115, CAL263, PI-103, GNE-477, CUDC-907, AEZS-136, and miRNA targeting the phosphoinositide 3 kinase gene (e.g., miR-7). In particular embodiments, the PKB inhibitor is selected from the group consisting of: VQD-002, perifosine, miltefosine, AZD5363, and MK-2206.

### DESCRIPTION OF THE FIGURES

FIGS. 1A-H. Shows results from Example 1 regarding either increased TEWL (transepithelial water loss) or sodium concentration augmented sodium flux in HESCs and keratinocyte cultures. As described in Example 1, HESCs or stratified keratinocytes (HaCaT) were cultured for 16 hours in the indicated conditions and ion fluxes were measured immediately after the samples were submerged in the bathing buffer. Sodium flux in HESCs culture with increased water loss (A) or increased media sodium concentration (10% more than control) (B). Sodium flux in stratified keratinocytes culture with increased water loss (C) or sodium concentration (D). Calcium (E) or chloride (F) flux in stratified keratinocytes culture with increased water loss. (G) Sodium flux in amiloride treated stratified keratinocytes culture with increased water loss. (H) Sodium flux in ENaC-knockdown stratified keratinocytes culture with increased water loss or sodium concentration. Background readings were obtained more than 1000 μm away from the sample. *p<0.05, **p<0.01.

FIGS. 2A-F. Regulation of COX-2 and PGE₂ by sodium flux is mediated by ENaC. Figures A and B show mRNA expression analysis of COX-2 as described in Example 1. (A) COX-2 mRNA expression in HESCs. HESCs were cultured with increased water loss condition for 4 or 16 hours (n=4). Total RNAs were isolated and RT-qPCR was performed. Gene expression was normalized according to the expression level of GAPDH. The level of COX-2 expression in HESCs treated with increased water loss was compared to that in control cells, which was set at 100% as expression level. (B) COX-2 mRNA expression keratinocytes culture. Stratified wild type or ENaC-knockdown HaCaT cells were cultured with increased water loss or sodium concentration condition for 16 hours (n=4). (C) PGE₂ protein expression. Monolayer wild type or ENaC-knockdown HaCaT cells were cultured with increased sodium concentration condition for 16 hours (n=4). The amount of PGE₂ in culture medium was measured by ELISA. (D) Western blot analysis (n=4). Stratified HaCaT culture was treated with increased water loss (labeled as A) or sodium concentration (labeled as N) as well as control (labeled as C). After 10, 30, 60, 240 minutes treatment, samples were harvested and western blot was performed with antibodies against phosphorylated-Akt, and Akt. β-actin was detected as a loading control. Figures E and F show the effect of the inhibition of PI3K/Akt signaling pathway on COX-2 expression. LY294002, a specific inhibitor for PI3K, was treated (10 μg/ml) in human foreskin keratinocytes (HK) culture for 16 hours with increased water loss (E) or sodium concentration medium (F) as well as control. Cox-2 mRNA (E) and PGE₂ (F) protein expression were analyzed by RT-qPCR and ELISA. *p<0.05, **p<0.01.

FIGS. 3A-C. ENaC senses sodium concentration changes in keratinocytes and regulates downstream genes. Stratified HaCaT and fibroblast were co-cultured in transwell plates and subjected to increased water loss and control conditions for 16 hours. Expression of α-SMA and pro-Col I was detected in dermal fibroblasts (HDF) with their specific antibodies and visualized with fluorescence labeled secondary antibodies, Nuclei were stained with DAPI. (FIG. 3A, Panels A-D) Stratified HaCaT and fibroblasts in control condition. (FIG. 3A, Panels E-H) Stratified HaCaT and fibroblasts in increased water loss condition. (FIG. 3A, Panels I-L) Stratified ENaC knockdown HaCaT and fibroblasts in control condition. (FIG. 3A, Panels M-P) Stratified ENaC knockdown HaCaT and fibroblasts in increased water loss condition. (FIGS. 3B-C) The quantification of expression levels of α-SMA and pro-Col I by ImageJ. The scale bar represents 100 μm.*p<0.05, **p<0.01.

FIGS. 4A-C. Show results from Example 1 where it was determine that PGE₂ is important for the activation of fibroblasts by keratinocytes. As described in Example 1, condition medium of stratified wild type (or ENaC knockdown) HaCaT culture under increased water loss was collected and supplemented to fibroblasts for 24 hours. Expression of α-SMA and pro-collagen I in dermal fibroblasts was tested as described in FIG. 3. (FIG. 4A, Panels A-D) Wild type HaCaT condition medium (CM). (FIG. 4A, Panels E-H) ENaC-knockdown HaCaT condition medium. (FIG. 4A, Panels I-L) ENaC-knockdown HaCaT condition medium plus 10 μM of PGE₂. (FIGS. 4B-C) Quantification of expression levels of α-SMA (FIG. 4B[[M]]) and pro-Col I (FIG. 4C[[N]]) was performed by ImageJ. The scale bar represents 100 μm. **p<0.01.

FIGS. 5A-H. Show results from Example 1 which demonstrated the reduction of hypertrophic scar formation by pharmacological inhibition of ENaC and COX-2 in vivo. As described in Example 1, full thickness 7 mm excision wounds were made on the ventral side of the rabbit ears at POD0. Amiloride (an ENaC inhibitor) or Celecoxib (a COX-2 inhibitor) was applied to the wounds every other day for a total of three times starting at POD14. Wounds harvested at POD28 and histological analysis was performed. (A-D) Representative pictures of wound histology at POD28. Rabbit ear wounds with Amiloride treatment (B) and control (A) and with Celecoxib treatment (D) and its control (C) are shown. (E) The scar elevation index (SEI) was calculated and used to evaluate the scar formation. (F. G) SEI. Twelve wounds for each dose of treatment and 12 controls were used for the analysis. *P<0.05. Scar bar stands for 1000 μm. **p<0.01. FIG. 5H shows a hypothetical mechanism of activation of fibroblast through ENaC by TEWL. The present invention is not limited by such a mechanism and an understanding of the mechanism is not necessary to practice the present invention.

FIGS. 6A-N. Expression of Naₓ, ENaC, and Prss8. The expression of Naₓ was visualized by confocal microscope (A-H). (A) Abundant Naₓ channel protein can be visualized in wild type keratinocyte. The wild type cell was scanned from 3 different layers, upper (B), middle (C), and lower (D) layer. In contrast, the Naₓ knockdown keratinocyte showed decreased number of Naₓ proteins on the surface (E) compared to wild type. Similarly, at different layers of the Naₓ knockdown cell, the expressions of Naₓ are significantly less than the wild type (F vs. B, G vs. C, and H vs. D). Localization of Naₓ, ENaC, and Prss8 are done by immunofluorescent staining with antibodies. All three membrane proteins, ENaC (I), Prss8 (J and M), and Naₓ (L) are consistently expressed in suprabasal layer. All three proteins have very similar expression profiles in human skin (K and N).

FIGS. 7A-B. The sodium flux change of stratified keratinocyte with or without knockdown of Naₓ. The sodium flux of the stratified wild type keratinocytes was increased by the stimulation of 10% more sodium in the culture medium compared to the cells in control medium (A). However, knockdown of Naₓ diminished the sodium flux increase as seen in wild type cells (B).

FIGS. 8A-H. Expression of Prss8, COX-2, IL-1β, and IL-8 in keratinocytes. In wild type keratinocytes, Prss8, COX-2, IL-1β, and IL-8 were all up-regulated by increased concentration of sodium (A-D). Prss8 was up-regulated at as early as 4 hours post stimulation (A). The other three genes also showed up-regulation at 4 hours post stimulation, however, more significant increase appeared at 16 hours post stimulation (B-D). Compared to wild type keratinocytes, knockdown of Naₓ down-regulated the expression of Prss8 (E), COX-2 (F), IL-1β (G), and IL-8 (H), while knockdown of ENaC only inhibited the expression of COX-2 (F) in response to high sodium stimulation.

FIGS. 9A-I. Prss8 is an important protein downstream of Naₓ. Knockdown of Prss8 dramatically decreased the expression levels of COX-2, IL-1β, and IL-8 with the stimulation of high concentration of sodium (A). Incubation the wild type keratinocytes with 10 μg/ml trypsin (a homolog to Prss8) has significantly increased the expressions of COX-2 (B), IL-1β (C), and IL-8 (D). Trypsin also recovered the up-regulation of IL-1β (F) and IL-8 (G) in both Naₓ and ENaC knockdown keratinocytes in response to high concentration of sodium stimulation. However, trypsin only recovered the up-regulation of COX-2 in Naₓ knockdown cells but not in ENaC knockdown cells under the stimulation of high concentration of sodium (E). As a primary function, Prss8 can activate ENaC by cleaving its α subunit. The cleaved and non-cleaved bands can be visualized by an ENaC specific protein with western blotting (H). Knockdown of Naₓ sufficiently inhibited the cleavage of the ENaC α subunit. However, the cleavage of the ENaC α subunit was enhance by trypsin in Naₓ knockdown keratinocytes (H and I).

FIG. 10. The keratinocytes differentiation with or without knockdown of Naₓ. Keratinocytes can be differentiated in differentiation medium to form a multiple cell layer structure like skin epidermis (Panel A). The keratinocyte differentiation marker K14 and K10 was seen in basal layer and suprabasal layer of differentiated keratinocytes, respectively (Panel D). Knockdown of Naₓ inhibited the keratinocyte differentiation (Panel B) with the absence of K10 marker in the cell (Panel E). Daily incubation with differentiation medium containing 10 ug/ml trypsin re-established the differentiation of keratinocytes (Panel C). The expression of differentiation markers are similar to the differentiated wild type keratinocytes.

FIGS. 11A-C. TEWL activated co-cultured dermal fibroblasts through the Naₓ on keratinocytes. Co-cultured with fully hydrated wild type stratified keratinocytes (HaCaT), the dermal fibroblast cells were activated upon the TEWL in keratinocytes, which was indicated by the increase of α-SMA (FIG. 11A, Panel D) and pro-collegan I (FIG. 11A, Panel E) compared to the fibroblast cells co-cultured with fully hydrated keratinocytes (FIG. 11A, Panel A and B, respectively). However, with the knockdown of Naₓ in keratinocytes, the activation of the co-cultured dermal fibroblasts dramatically decreased even with (FIG. 11A, Panel J and K) or without (FIG. 11A, Panel G and H) the stimulation of TEWL on keratinocytes. The statistical analyses with the signal intensity of the two markers in different dermal fibroblast cells showed that the knockdown of Naₓ in epidermal keratinocytes significantly decreased the activation of the co-cultured dermal fibroblast in both control and TEWL conditions (FIGS. 11B-C).

FIG. 12. An exemplary schematic flow of the hypothetical pathways of Naₓ. The Naₓ on keratinocyte can sensing a slight change of sodium influx with the stimulation of skin damaging including wounding and some skin diseases. This small change of the sodium flux causes the phosphorylation of Naₓ, which sequentially leads to the activation of CAP-1/Prss8 through stress-activated protein kinase (SAPK) (1). The activation of Prss8 in human keratinocytes can activate two membrane proteins, ENaC (2), and PAR (3). The ENaC has been reported in our previous study that can activate the synthesis of COX-2 through PI3K/Akt (4). The PAR2 pathway, which is widely reported in many other cell systems, can activate the expressions of several other inflammatory factors, such as IL-1β and IL-8 in this case (5 and 6). The references for this Naₓ pathway include: (1) Kanke, et al, 2001. J Biol Chem 278 (84), 31657-31666. (2) Rossier and Stutts, 2009. Annu Rev Physiol 71, 361-379. (3) Frateschi et al 2011, Nat Comms 2, 161. (4) Ruan et al 2012, Nat Med 18, 1112-1117. (5) Rattenholl, et al, 2007. J Invest Dermatol 127, 2245-2252. (6) Julovi, et al 2011, Am J Pathol 179, 2233-2242; all of which are herein incorporated by reference in their entireties.

FIGS. 13A-C. Expression of S100A8 (FIG. 13A), A9 (FIG. 13B) and A12 (FIG. 13C) with the regulation of ENaC and Naₓ. In wild type keratinocytes, the expression levels of S100A8, S100A9, and S100A12 were all up-regulated upon the stimulation of 10% increase of sodium in the culture medium. Knockdown of Naₓ on keratinocytes completely abolished the up-regulation of these three genes with the induction of high concentration of sodium. The knockdown of ENaC in keratinocytes also attenuate the up-regulation of S100A8, A9, and A12 in response to high concentration of sodium stimulation compared to wild type keratinocytes, however the up-regulations were not completely eliminated. This data indicates that both ENaC and Naₓ can modify the gene expression patterns of S100A8, A9, and A12, while the Naₓ appeared to be more upstream and regulating multiple pathways to alter their gene expressions.

### DETAILED DESCRIPTION

The present invention provides methods of reducing cutaneous scar formation by treating a cutaneous wound with a composition comprising a therapeutic agent that is a sodium channel blocker and/or an inhibitor of the Naₓ/SCN7A pathway. The present invention also provides wound cover components impregnated with such compositions, kits composed of such compositions with a wound dressing or sterile wipe, and mixtures of such compositions with a topical component (e.g., cream, ointment, or gel) suitable for application to a cutaneous wound. The present invention also provides compositions, kits, devices, and methods for treating skin conditions (e.g., dermatitis, psoriasis, or other skin conditions) with such compositions and devices. Examples of such therapeutic agents include, but are not limited to, an inhibitor of a gene or protein selected from: ENac, COX-2, PGE2, PI3K, PKB, Naₓ Prss8, IL-1β, IL-8, SAPK, Erk gene, p38 gene, PAR2, S100A8, S100A9, S100A12. Although it is known that the inflammatory response which results from disruption epithelial barrier function after injury results in excessive scarring, the upstream signals were unknown. Epithelial disruption results in transepithelial water loss (TEWL). In developing embodiments of the present invention, it was hypothesized that epithelial cells can sense TEWL via changes in sodium homeostasis and sodium flux into keratinocytes. In developing embodiments of the present invention, it was also hypothesized that these changes in sodium flux result in activation of pathways responsible for keratinocyte-fibroblast signaling, and ultimately lead to activation of fibroblasts. In the Examples presented below, it was demonstrated that perturbations in epithelial barrier function lead to increased TEWL and increased sodium flux in keratinocytes. It was identified that sodium flux in keratinocytes is mediated by epithelial sodium channels (ENaC). It was also demonstrated that activation of ENaC cause increased secretion of proinflammatory cytokines and activation of fibroblast via the COX-2/prostaglandin E₂ (PGE₂) pathway. Similar changes in signal transduction and sodium flux occur by increasing the sodium concentration in the media in epithelial cultures, or human ex vivo skin cultures (HESC) in vitro. Blockade of ENaC activation, prostaglandin synthesis by COX-2, or PGE₂ receptors all reduce markers of fibroblast activation and collagen synthesis. In addition, as described in the Examples below, employing a validated in vivo excessive scar model in the rabbit ear, it was demonstrated that utilization of either an ENaC sensitive sodium channel blocker or a COX-2 inhibitor results in a marked reduction in scarring. Other experiments demonstrated that the activation of COX-2 in response to increase sodium flux is mediated through the PIK3/Akt pathway. While the present invention is not limited to any particular mechanism, and an understanding of the mechanism is not necessary to practice the invention, these results appear to indicate that ENaC responds to small changes in sodium concentration with inflammatory mediators, and suggests that ENaC pathway is a potential target for a novel strategy to prevent fibrosis.

Work conducted during the development of embodiments of the present invention showed successful knocked down of the expression of Naₓ, Prss8, and ENaC with shRNA in human keratinocytes (HK). The shRNA sequences employed were as follows:

siRNA Naₓ:
Forward:
(SEQ ID NO: 1)
CCGGGCTGACATGATCTTTACTTATCTCGAGATAAGTAAAGATCATGTC
AGCTTTTTG-;
Reverse:
(SEQ ID NO: 2)
AATTCAAAAAGCTGACATGATCTTTACTTATCTCGAGATAAGTAAAGAT
CATGTCAGC;
siRNA ENaC-α:
Forward:
(SEQ ID NO: 3)
CCGGCGATGTATGGAAACTGCTATACTCGAGTATAGCAGTTTCCATACA
TCGTTTTTG;
Reverse:
(SEQ ID NO: 4)
AATTCAAAAACGATGTATGGAAACTGCTATACTCGAGTATAGCAGTTTC
CATACATCG;
siRNA Prss8:
Forward:
(SEQ ID NO: 5)
CCGGGTGGCCATTCTGCTCTATCTTCTCGAGAAGATAGAGCAGAATGGC
CACTTTTTG;
Reverse:
(SEQ ID NO: 6)
AATTCAAAAAGTGGCCATTCTGCTCTATCTTCTCGAGAAGATAGAGCAG
AATGGCCAC.

In work conducted during the development of embodiments of the present invention, three downstream genes, Cyclooxygenase 2 (COX-2), Interleukin-1β (IL-1β), and Interleukin 8 (IL-8) were found related to this Naₓ-Prss8-ENaC pathway. As a consequence of the ENaC knockdown in HK, one of the important inflammatory factors, COX-2 was significantly down-regulated with the knockdown of Naₓ, Prss8, or ENaC in keratinocytes compared to the wild type during increased water loss. However, the expressions of the other two cytokines, IL-1β and IL-8 were only down-regulated with the knockdown of Naₓ or Prss8, but not ENaC. This suggests that Naₓ may initiate two different pathways. One is to regulate the expressions of IL-1β and IL-8. The other pathway is through activation of ENaC and further regulates the expression of COX-2. Both pathways were conducted by Prss8 following the Nax.

To further confirm the pathway of Naₓ-Prss8-ENaC, a Prss8 homolog, trypsin was used to activate the ENaC with the knockdown of Naₓ. With the knockdown of Naₓ, keratinocytes failed to be activated by cell differentiation medium. Additional trypsin in the differentiation medium successfully recovered the differentiation of Naₓ knockdown cells. Meanwhile, the additional trypsin also up-regulated the expression of COX-2, IL-1β, and IL-8 in the Naₓ knockdown keratinocytes under the stimulation of high concentration of sodium. The results suggested that Naₓ can conduct the activation of ENaC and the other pathway through Prss8.

In work conducted during the development of embodiments of the present invention, co-cultured with human dermal fibroblasts (HDF), the wild type HK dramatically activated the HDF with increased sodium concentration. However, Naₓ knockdown HK failed to activate most of the co-cultured HDF with the increased sodium concentration. Since the HDF is the main player of skin hypertrophic scar formation, this suggests the importance of HK Naₓ in determination of scar hypertrophy in wound healing.

Inhibitors of Prss8 (e.g., human Prss8) mRNA or protein include, but are not limited to, aprotinin, antipain, leupeptin, benzamidine, hepatocyte growth factor activator gene or protein inhibitor 1 (HAI-1), antibodies directed toward the Prss8 protein (e.g., a monoclonal antibody directed toward the human protein), and siRNA or miRNA directed toward Prss8 mRNA.

Inhibitors of epidermal sodium channel (ENac) (e.g., human ENac) mRNA or protein include, but are not limited to, amiloride, triamterene, benzamil, GS9411, P-365, pyrazine derivatives, antibodies directed toward the ENac protein (e.g., a monoclonal antibody directed toward the human protein), and siRNA or miRNA directed toward ENac mRNA.

Inhibitors of cyclooxygenase-2 (COX-2) (e.g., human COX-2) mRNA or protein include, but are not limited to, celecoxib (Celebrex), valdecoxib (Bextra), rofecoxib (Vioxx), diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, tolmetin, acetaminophen, antibodies directed toward the COX-2 protein (e.g., a monoclonal antibody directed toward the human protein), and siRNA or miRNA directed toward ENac mRNA.

Inhibitors of prostaglandin E2 (PGE2) (e.g., human PGE2) mRNA or protein include, but are not limited to, curcumin, SC-560, AH6809, sulforaphane, wagonin, rifampin, antibodies directed toward the PGE2 protein (e.g., a monoclonal antibody directed toward the human protein), and siRNA or miRNA directed toward PGE2 mRNA.

Inhibitors of phosphoinositide 3 kinase (PI3K) (e.g., human PI3K) mRNA or protein include, but are not limited to, LY294002, Wortmannin, demethoxyviridin, Perifosine, CAL101, PX-866, IPI-145, BAY 80-6946, BEZ235, TGR 1202, SF1126, INK1117, GDC-0941, BKM120, XL147, XL765, Palomid 529, GSK1059615, ZSTK474, PWT33597, IC87114, TG100-115, CAL263, PI-103, GNE-477, CUDC-907, AEZS-136, antibodies directed toward the PI3K protein (e.g., a monoclonal antibody directed toward the human protein), and siRNA or miRNA directed toward PI3K mRNA.

Inhibitors of protein Kinase B (PKB or Akt) (e.g., human PKB) mRNA or protein include, but are not limited to, VQD-002, perifosine, miltefosine, AZD5363, and MK-2206, antibodies directed toward the PKB protein (e.g., a monoclonal antibody directed toward the human protein), and siRNA or miRNA directed toward PKB mRNA.

Inhibitors of Naₓ (SCN7A) (e.g., human Nax) mRNA or protein include, but are not limited to, antibodies directed toward the Naₓ protein (e.g., a monoclonal antibody directed towards the human protein), and siRNA or miRNA directed toward Naₓ mRNA (see SEQ ID NOs: 1 and 2, and those described in Ke et al., Neuroscience, 2012, Dec. 27:227:80-9, herein incorporated by reference).

Inhibitors of interleukin-1β (IL-1β) (e.g., human IL-1beta) mRNA or protein include, but are not limited to, Canakinumab (Ilaris) or other antibodies directed toward the IL-1β protein (e.g., a monoclonal antibody directed toward the human protein), and siRNA or miRNA directed toward IL-1β mRNA (e.g., as described in Peng et al., Glia. 2006 Nov. 1; 54(6):619-29, herein incorporated by reference).

Inhibitors of interleukin 8 (IL-8) (e.g., human IL-8) mRNA or protein include, but are not limited to, Reparixin, molecules disclosed in U.S. Pat. No. 6,448,379 (herein incorporated by reference), the peptide Ac-Arg-Arg-Trp-Trp-Cys-Arg-NH2 (SEQ ID NO: 7), antibodies directed toward the IL-8 protein (e.g., a monoclonal antibody directed toward the human protein), and siRNA or miRNA directed toward IL-8 mRNA (see, e.g., Merritt et al., JNCI J Natl Cancer Inst Volume 100, Issue 5, Pp. 359-372, herein incorporated by reference).

Inhibitors of SAPK (e.g., human SAPK) mRNA or protein include, but are not limited to, SP600125, DJNK11, antibodies directed toward the SAPK protein (e.g., a monoclonal antibody directed toward the human protein), and siRNA or miRNA directed toward SAPK mRNA (see e.g., Shen et al., Cell Biochem Biophys. 2012 September; 64(1):17-27, herein incorporated by reference).

Inhibitors of Erk (e.g., human Erk) mRNA or protein include, but are not limited to, SCH772984, FR180204, AEZS-131, PD98059, antibodies directed toward the Erk protein (e.g., a monoclonal antibody directed toward the human protein), and siRNA or miRNA directed toward Erk mRNA.

Inhibitors of p38 (e.g., human p38) mRNA or protein include, but are not limited to, SB203580, LY2228820, SC-68376, VX-745, antibodies directed toward the p38 protein (e.g., a monoclonal antibody directed toward the human protein), and siRNA or miRNA directed toward p38 mRNA (see e.g., Chen et al., Cancer Res Dec. 1, 2009 69; 8853; herein incorporated by reference).

Inhibitors of PAR2 (e.g., human PAR2) mRNA or protein include, but are not limited to, K-12940, K-14585, P2pal-18S, antibodies directed toward the PAR2 protein (e.g., a monoclonal antibody directed toward the human protein), and siRNA or miRNA directed toward PAR2 mRNA (see, e.g., Lin et al., Int J Biochem Cell Biol. 2008; 40(6-7): 1379-1388).

Inhibitors of S100A8 (e.g., human S100A8) mRNA or protein include, but are not limited to, arachidonic acid, antibodies directed toward the S100A8 protein (e.g., a monoclonal antibody directed toward the human protein), and siRNA or miRNA directed toward S100A8 mRNA.

Inhibitors of S100A9 (e.g., human S100A9) mRNA or protein include, but are not limited to, arachidonic acid, quinoline-3-carboxamide compounds, antibodies directed toward the S1009 protein (e.g., a monoclonal antibody directed toward the human protein), and siRNA or miRNA directed toward S100A9 mRNA.

Inhibitors of S100A12 (e.g., human S100A12) mRNA or protein include, but are not limited to, epigallocatechin-3-gallate (EGCG), antibody ab37657, or other antibodies directed toward the S10012 protein (e.g., a monoclonal antibody directed toward the human protein), and siRNA or miRNA directed toward S100A12 mRNA.

The therapeutic agents of the present invention may be formulated in compositions for topical administration, such as in ointments, creams, suspensions, lotions, powders, solutions, pastes, gels, sprays, aerosols or oils. In certain embodiments, topical formulations comprise patches or dressings such as a bandage or adhesive plasters impregnated with the therapeutic agent, and optionally one or more excipients or diluents. In some embodiments, the topical formulations include a compound(s) that enhances absorption or penetration of the therapeutic agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. In some embodiments, oily phase emulsions of this invention are constituted from known ingredients in an known manner. This phase typically comprises a lone emulsifier (otherwise known as an emulgent), it is also desirable in some embodiments for this phase to further comprises a mixture of at least one emulsifier with a fat or an oil or with both a fat and an oil.

In certain embodiments, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations. Emulgents and emulsion stabilizers suitable for use in the formulation of the present invention include, for example, Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

### EXAMPLES

### Example 1

### Scar Reduction Via Blocking ENaC or ENaC Signal Transduction Pathway

This Example describes experiments conducted to evaluate the mechanism by which TEWL (trans-epidermal water loss) leads to pro-inflammatory cytokine expression and increased scarring. It was hypothesized that changes in epithelial hydration and sodium homeostasis are monitored through ENaC (epithelial sodium channel) and that this protein regulates downstream inflammatory pathways leading to fibroblast activation. Remarkably, blocking ENaC or ENaC mediated signal transduction with a commercially available sodium channel blocker (amiloride) or a COX-2 inhibitor lead to significant improvement in scarring. Given that compromised barrier function with increased TEWL is a major factor in many types of inflammatory dermatitis, these targets are useful for many skin diseases.

Increased trans-epidermal water loss results in higher sodium flux.

To estimate the change of sodium flux of skin, the sodium flux was measured in human ex vivo skin culture (HESC), which retains important elements of in vivo skin and recapitulates the features of human wound repair (25, 26). The HESCs were cultured in an air/liquid interface after tape-stripping the epithelium to remove the stratum corneum and impair the barrier function of skin. Conditions to increase TEWL were created by placing the cultured HESCs in an air-flow chamber with reduced humidity. Control HESCs were cultured in closed chamber which maintains a humid environment. To validate increased TEWL, expression levels of aquaporin 3 (AQP3) were measured, which has been reported as a molecular marker to indicate the hydration conditions of skin (27, 28). Western blot analysis showed that expression of AQP3 protein was increased by 80% in cultured HESC cells placed in the dry air-flow chamber over controls. This study was replicated in stratified keratinocyte cultures (HaCaT) to validate results. Conditions to promote water loss from keratinocytes were created by exposing the surface of cultured cells to air-flow. Control stratified keratinocyte cultures were kept in a liquid environment of standard media. Both mRNA and protein expression analysis showed enhanced expression of AQP3 by increased TEWL in stratified keratinocytes culture.

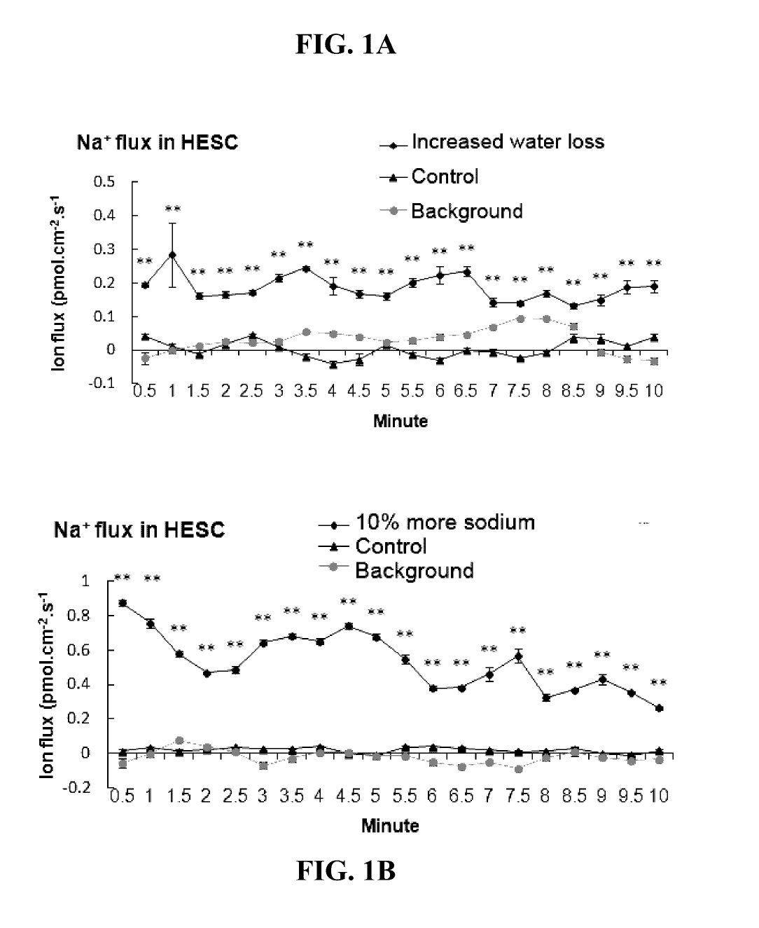

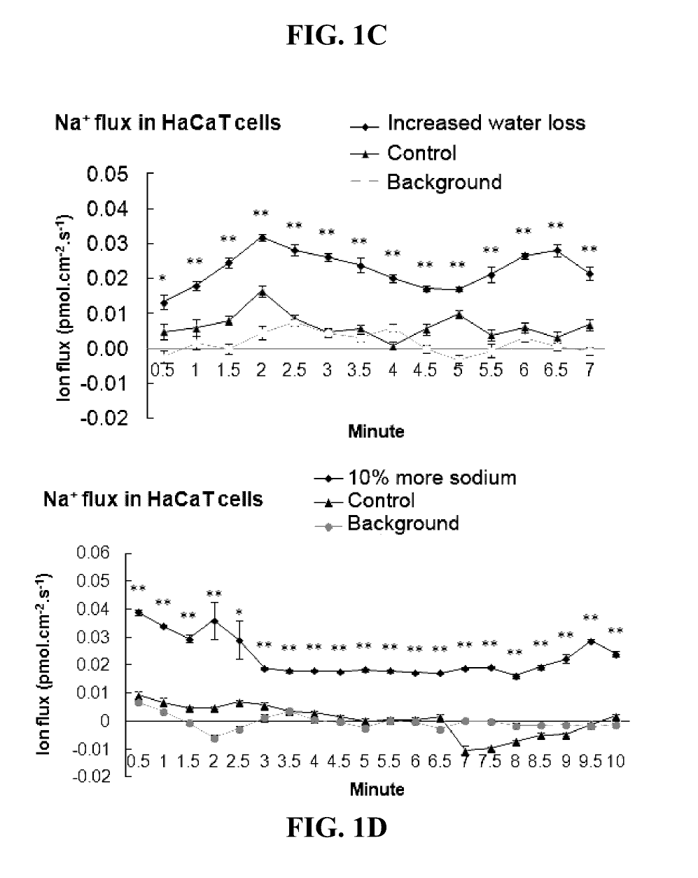

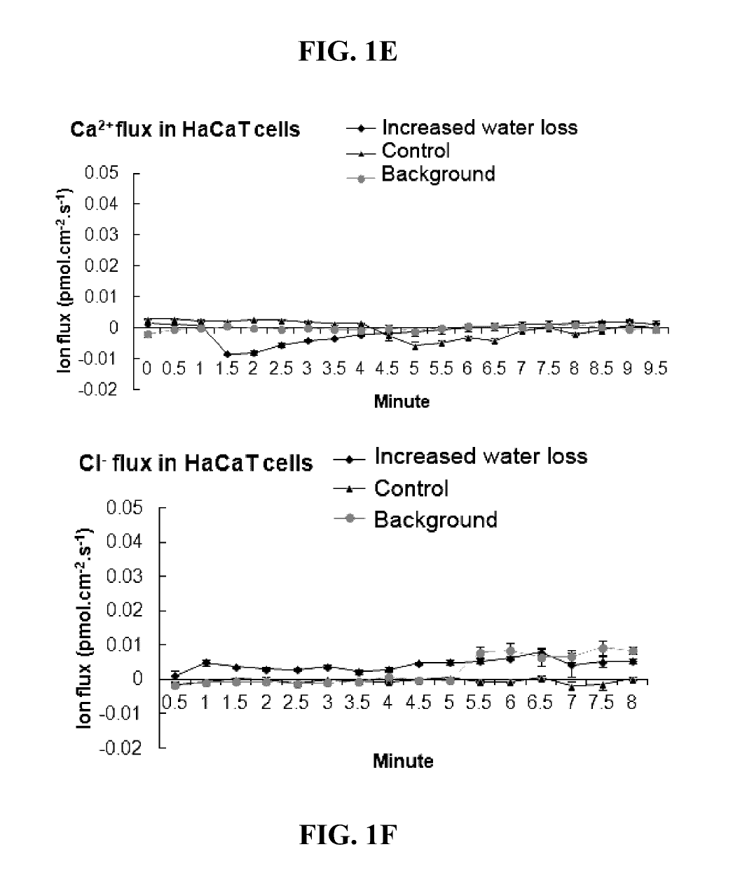

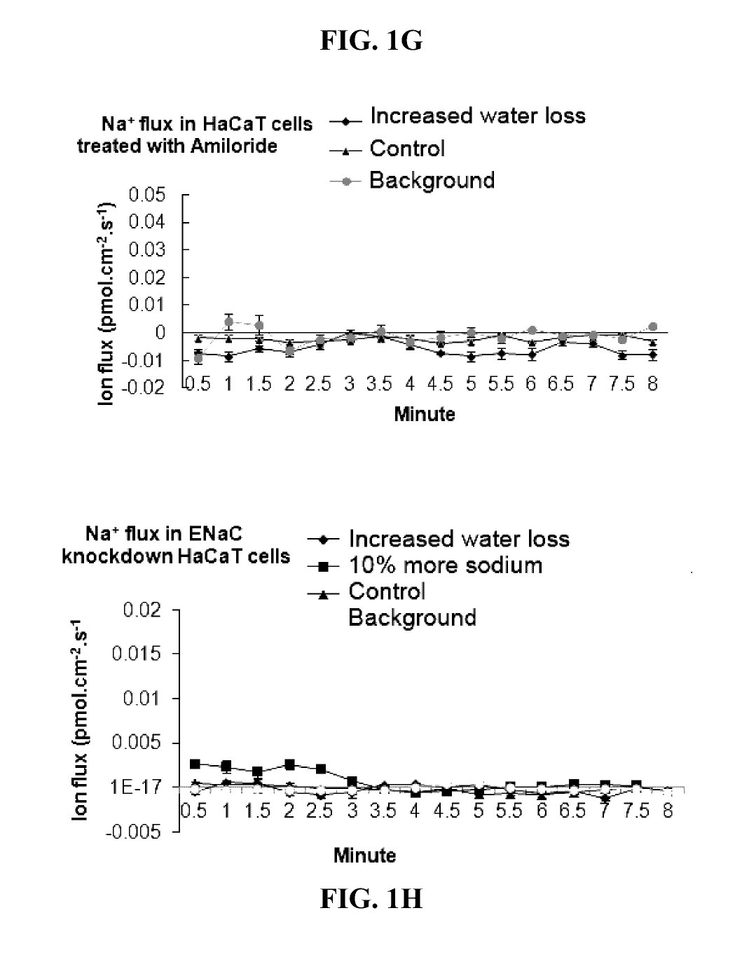

Changes in sodium flux were evaluated using a scanning ion-selective electrode technique (SIET). HESCs placed in air-flow chambers demonstrated increased sodium flux compared to control HESCs (FIG. 1A). Similarly, the sodium flux of stratified keratinocyte cell line, HaCaT, showed a significant increase in response to increased water loss (>0.02 pmol·cm-2·s-1) (FIG. 1C). Interestingly, higher sodium flux was found in HESC compared to stratified keratinocyte cultures (compare FIG. 1A vs. 1C), which is possibly due to the different characteristics of the two samples.

Water loss may also be modeled by increasing the concentration of sodium in the culture medium and such a study was conducted to confirm the results above. Sodium concentration was increased in the culture medium, which contains 110 mM sodium, by 10% (11 mM), to 121 mM sodium. Increasing sodium concentration in the culture medium enhanced the sodium flux in HESC and HaCaT cells by five and two fold, respectively, compared to controls (FIGS. 1B and 1D). To evaluate the flux and possible contribution of other ions, chloride and calcium were also evaluated, however, no detectable fluxes of Ca2+ and Cl− were found in the stratified keratinocyte culture by increased water loss (FIGS. 1E and 1F). These results suggest that either water loss or increasing extracellular sodium concentration increases sodium flux in skin epithelial cells.

### Increased Sodium Flux is Mediated Through ENaC and Enhances Expression of COX-2 and PGE2 in Keratinocyte Through PI3k/Akt Pathway

The amiloride-sensitive ENaC facilitates sodium absorption in many epithelia tissues, such as kidney (29) and lung (30). It was investigated whether ENaC mediates the changes in sodium sodium flux caused by increased water loss or increased sodium concentration. This was accomplished by evaluating changes in sodium flux after pharmacological blockade and gene knockdown of ENaC. Treatment of stratified keratinocytes with amiloride (10 μg/ml) completely abolished the enhanced sodium flux due to increased water loss (FIG. 1C vs. 1G). In addition, a loss of function study was performed of α-ENaC, by knockdown with shRNA. Stratified HaCaT cells with ENaC knockdown did not show increased sodium flux with either conditions of increased water loss or increased sodium (FIGS. 1C and 1D vs. 1H).

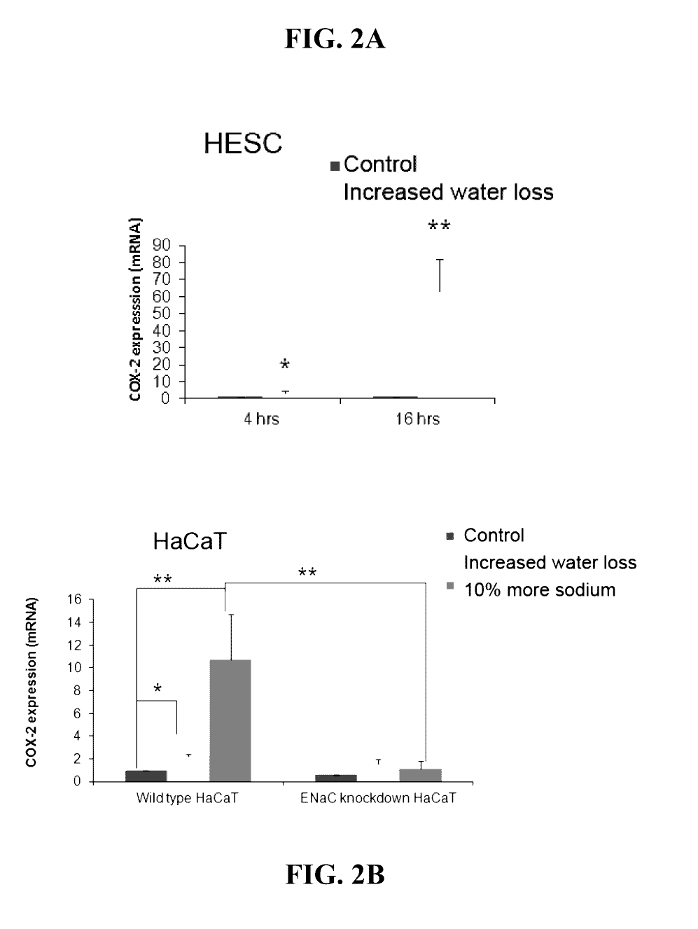

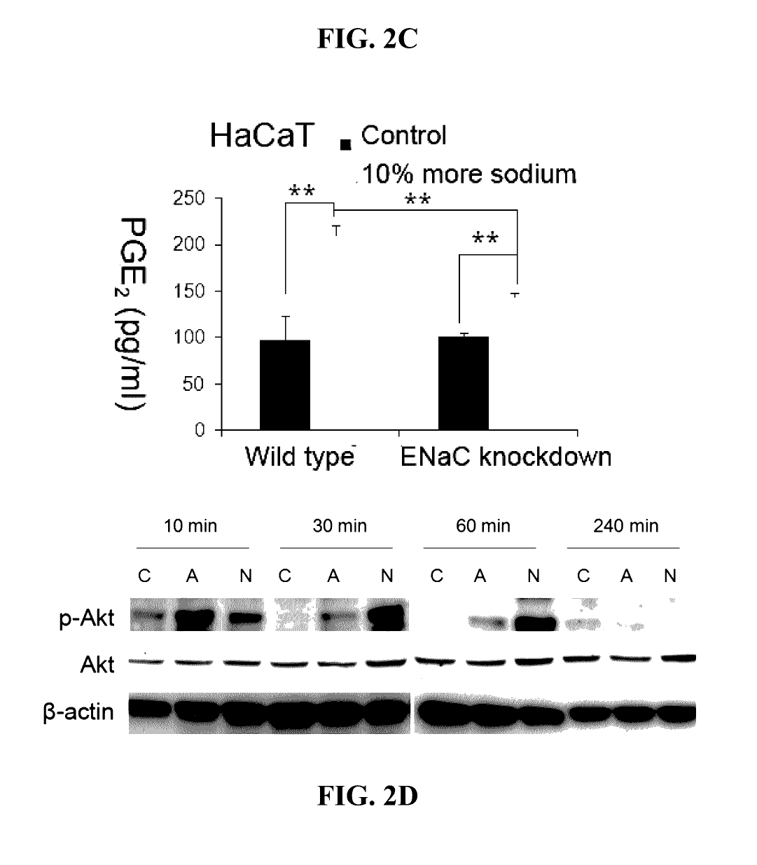

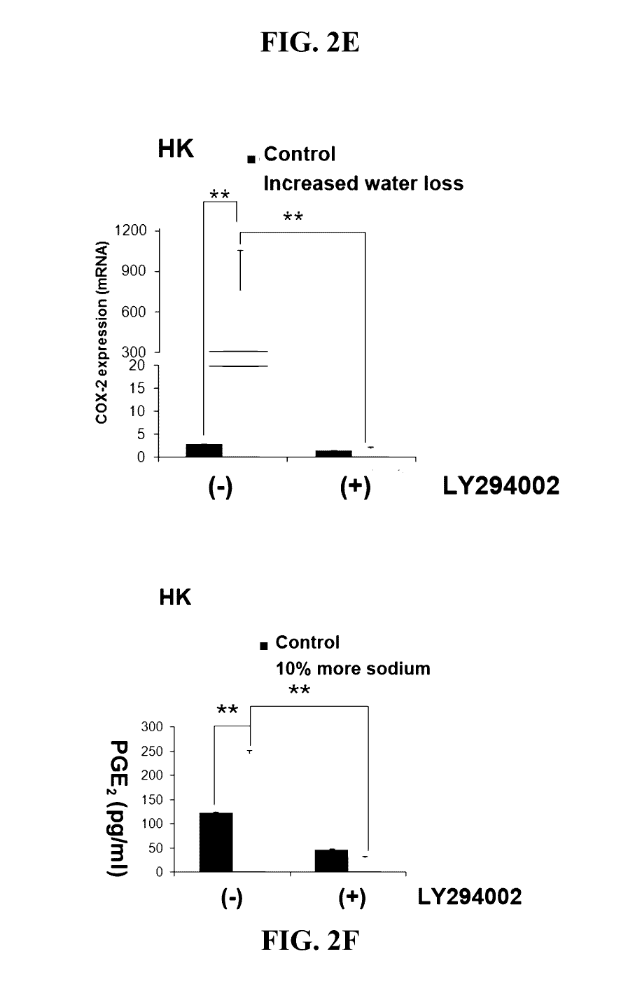

In contrast to mucosal wounds which heal in a liquid environment, cutaneous wounds have increased water loss. It was hypothesized that increased sodium flux associated with TEWL upon cutaneous injury results in up-regulation of inflammatory genes including COX-2 and the downstream end product prostaglandin E2 (PGE2). The expression of COX-2 mRNA in epidermis of HESC was increased by 3.3±1.7 (mean±s.e.m.) fold within 4 hours of increased water loss treatment (FIG. 2A). In the epidermal layer of HESC, COX-2 mRNA expression was increased to 63.1±19.3 fold in response to increased water loss at 16 hours compared to the control HESC. Such results were further confirmed in stratified keratinocyte cultures with an increase in both COX-2 mRNA and protein under conditions of increased water loss or increased sodium concentration (FIG. 2B).

ELISA was performed to quantify the amount of PGE2 secreted by cells into the culture medium (FIG. 2C). In wild type HaCaT cells, the amount of PGE2 released into culture medium in normal culture condition was 97.9±25.3 pg/ml. By adding increase sodium concentration in the media by 10%, the release of PGE2 increased to 211.0±10.6 pg/ml. In ENaC knockdown HaCaT cells, the high sodium concentration did not significantly increase the production of PGE2 (from 100.9±4.3 pg/ml in normal medium to 144.3±3.1 pg/ml in high sodium medium). In addition, it was investigated whether activation of the COX-2 inflammatory pathway was regulated via ENaC. This was tested by knock down of ENaC in HaCaT cells. The increase of COX-2 expression in response to increased water loss or sodium concentration in culture was dramatically diminished by knockdown of ENaC in HaCaT (FIG. 2B). The decrease of COX-2 in ENaC knockdown keratinocytes led to a significant reduction of PGE2. (FIG. 2C).

Previous reports have identified phosphorylation cascades that can activate COX-2 expression (32-34). In this Example, it was tested whether phosphorylation of Akt mediates the up-regulation of COX-2 expression and activity caused by increased sodium flux. Phosphorylation of these pathways was evaluated 10, 30, 60 and 240 minutes after HaCaT cells were treated with increased water loss or increased sodium concentrations. Phosphorylation of Akt was rapidly increased at 10 min with increased water loss or 10% higher sodium concentration in culture, and the increase lasted until 60 minutes post treatment (FIG. 2D).

It is known that phosphatidylinositol 3-kinases (PI3K) activation activates phosphorylation of Akt. This Example addressed whether PI3K/Akt signaling is involved in activation of the COX-2 pathway caused by increases in sodium flux. Treatment of PI3K inhibitor, LY294002, reduced the basal level COX-2 mRNA expression in human foreskin primary keratinocytes (HK) and PGE2 protein in culture medium (FIGS. 2E and 2F). Interestingly, robust induction of COX-2 in response to increased water loss and sodium concentration was drastically reduced by blockage of PI3K/Akt signaling pathway with LY294002 (FIG. 2E). As a consequence of COX-2 reduction, PGE2 release in the culture medium of HK was also reduced in the treatment condition (FIG. 2F).

### ENaC Senses Sodium Concentration Change in Epidermal Keratinocytes and Regulates Gene Expression in Dermal Fibroblasts

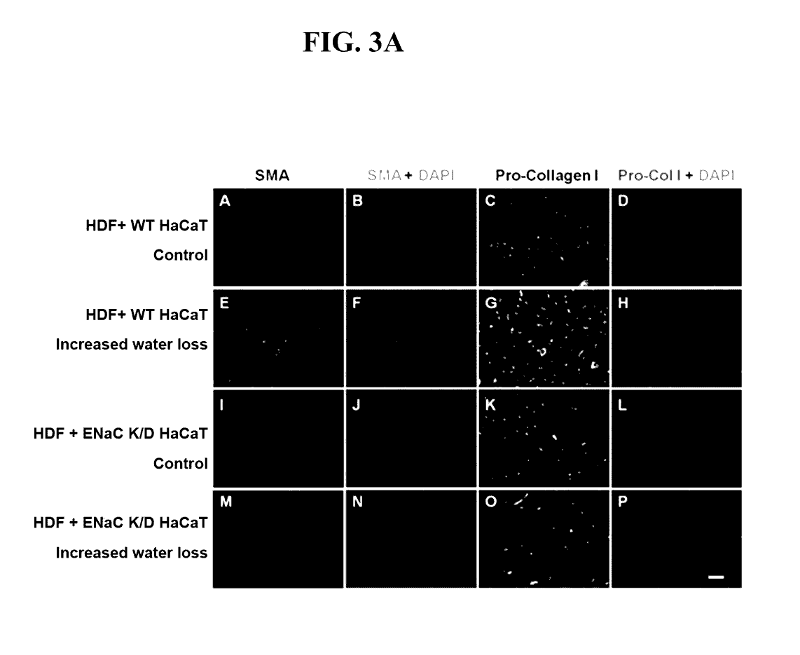

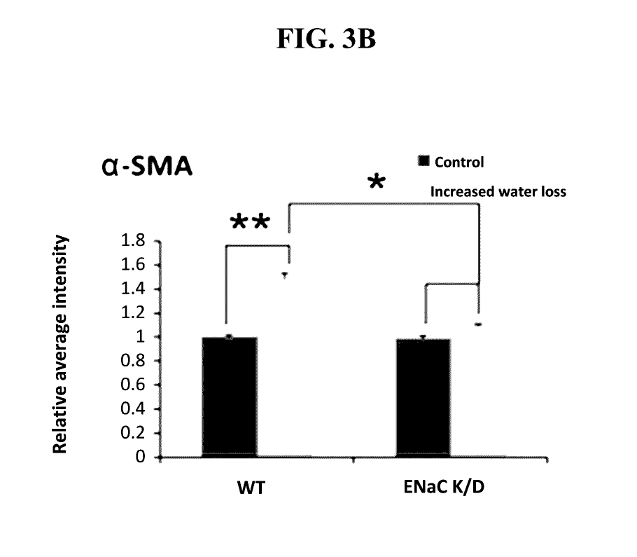

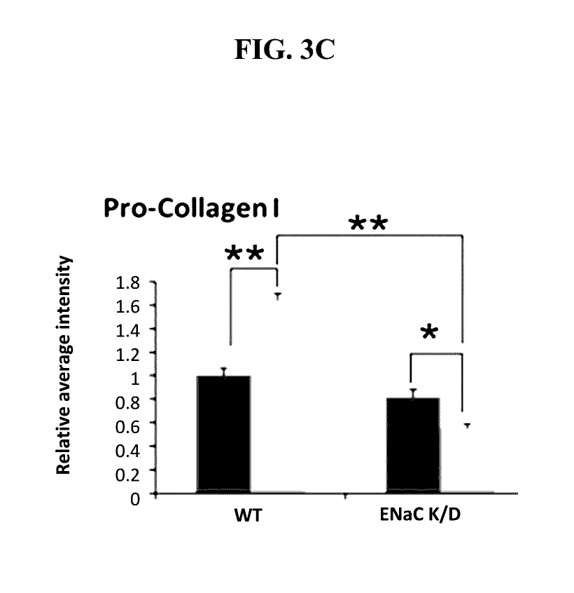

It was investigated whether ENaC is involved in this epidermal-dermal cross talk. Human dermal fibroblasts were co-cultured with stratified HaCaT cells on 6 well plates. Increased water loss was generated by placing stratified HaCaT air/liquid interface in a dry air flow chamber. In controls, both fibroblast and stratified HaCaT were submerged in culture medium. It is known that dermal fibroblasts show increased expression of alpha smooth muscle actin (α-SMA) and pro-collagen I (pro-Col I) upon activation (35, 36). Expression of α-SMA and pro-Col I in dermal fibroblast was significantly increased by water loss when compared to controls (FIG. 3A, Panels A-D vs. 3 A, Panels E-H). Quantification with ImageJ showed that expression of α-SMA and pro-Col was increased by 49±4% and 65±5%, respectively, by increased water loss (FIGS. 3B and 3C). Under control conditions, knocking out ENaC did not alter the expression of α-SMA and pro-Col when compared wild type HaCaT (FIGS. 3B and 3C). However, enhanced expression of α-SMA and pro-Col in fibroblast was not detected in water loss conditions when ENaC was knocked out with shRNA (FIG. 3A, Panels I-P, FIG. 3B, and FIG. 3C). These results suggest that ENaC is an important upstream protein for keratinocyte sodium sensing and for keratinocyte-fibroblast signaling via regulation of soluble mediators production.

These results also strongly suggest that the ENaC pathway is important for the COX-2 expression in response to sodium flux in keratinocytes (FIG. 2). To evaluate whether COX-2 is an important downstream mediator of ENaC in epidermal keratinocytes-dermal fibroblasts signaling, COX-2 was knocked down in HaCaT cells with shRNA. Activation of fibroblast was abolished when co-cultured with COX-2 knockout HaCaT in an increased water loss conditions.

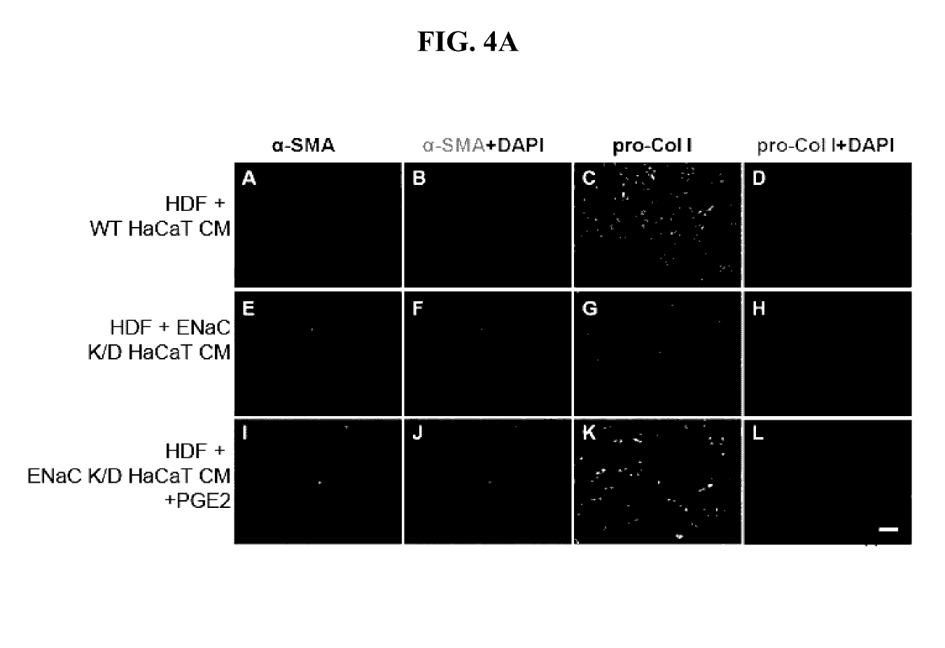

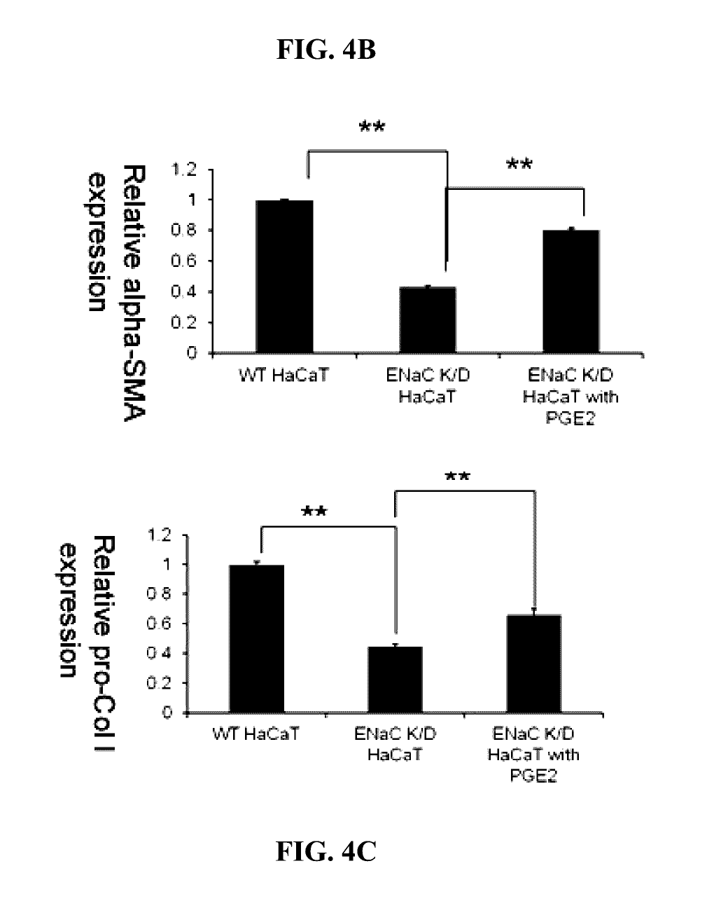

Since the PGE2 is a downstream product of COX-2 and found to be involved in regulation of fibroblast cytoskeletal dynamics during airway epithelium injury (37), it was hypothesized that PGE2 signaling is important for keratinocyte-fibroblast signaling. Prostaglandins EP2 and EP4, after secretion from keratinocytes, and since they can activate target cells via PGE2 receptors, making them suitable for cellular interactions. Compared to the wild type HaCaT culture condition medium, the ENaC knockdown HaCaT culture medium failed to activate the dermal fibroblasts (FIG. 4A, Panels A-H). Addition of 10 μM PGE2 into the ENaC knockdown HaCaT culture conditioned medium successfully rescued the activation of fibroblasts (FIG. 4A, Panels I-L). Estimation of amount of α-SMA and pro-Col I by ImageJ clearly showed the increase of both markers in co-cultured dermal fibroblasts after adding additional PGE2 into ENaC knockdown HaCaT culture conditioned medium. Next, antagonists against PGE2 receptor EP2 and EP4 were applied to block the effects of keratinocytes cultured under increased water loss conditions. Blockade of PGE2 receptors with AH6809 (EP2 receptor antagonist) and CAY10580 (EP4 receptor antagonists) in condition medium collected from the HaCaT with increased water loss reduced activation of fibroblasts to control levels (S3). Combination of AH6809 and CAY10580 further reduced activation of fibroblasts.

### Pharmacological Blockage of ENaC or COX-2 Reduces Cutaneous Hypertrophic Scar Formation in Animals.

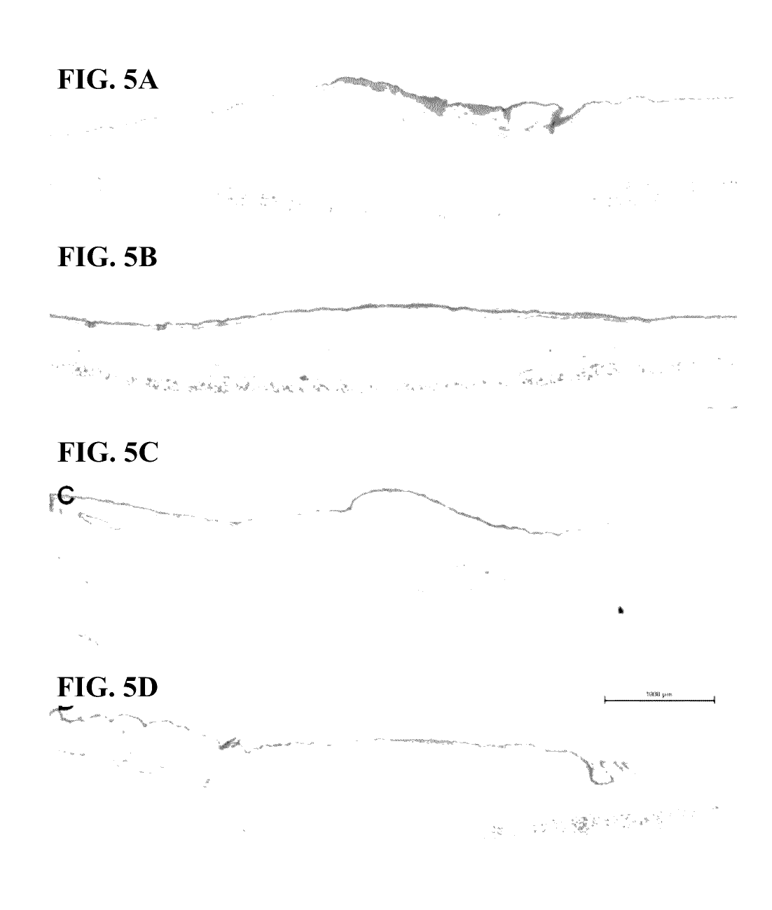

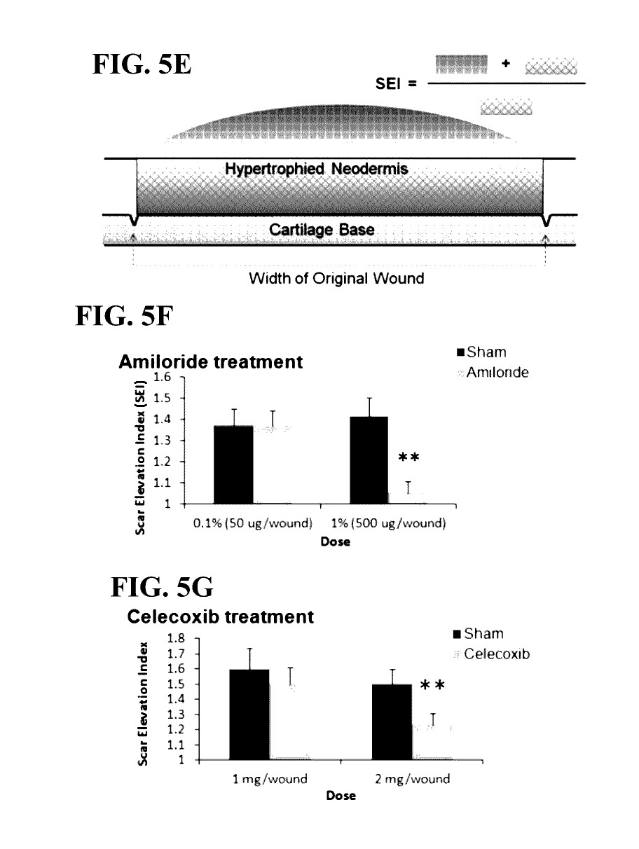

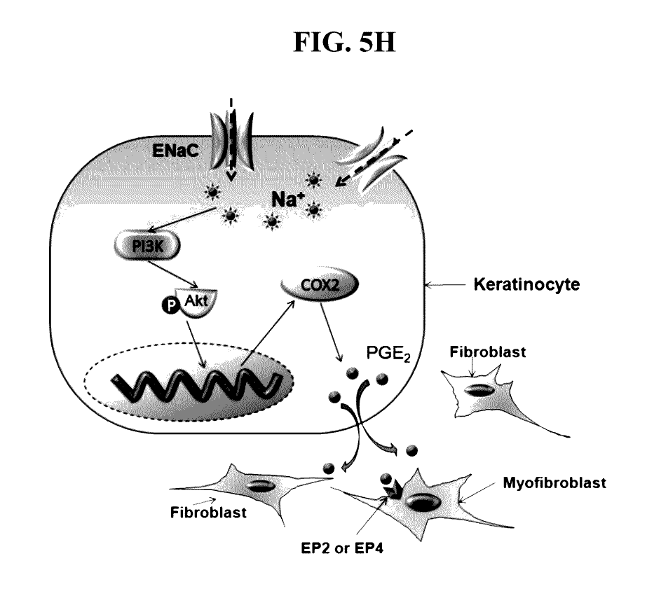

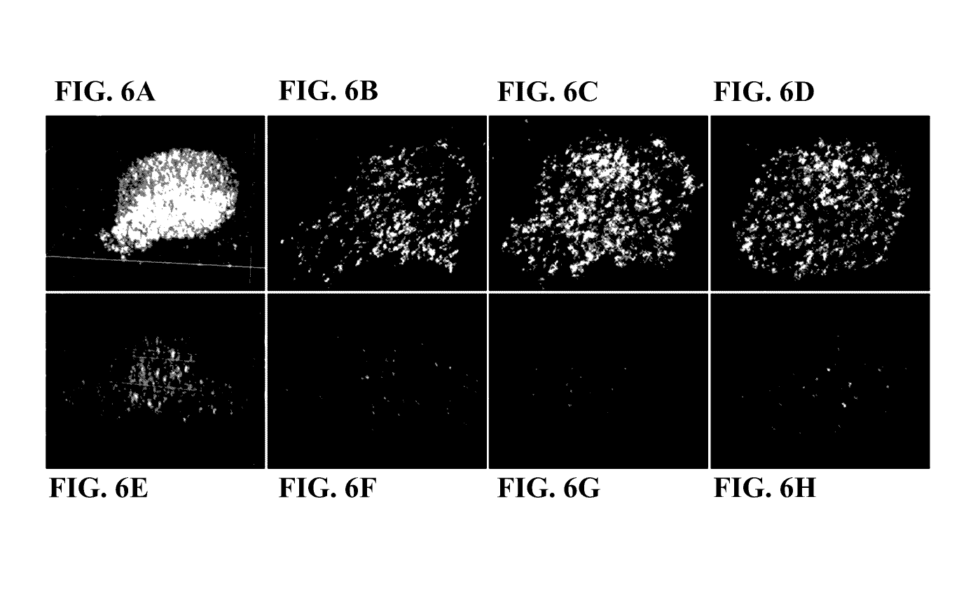

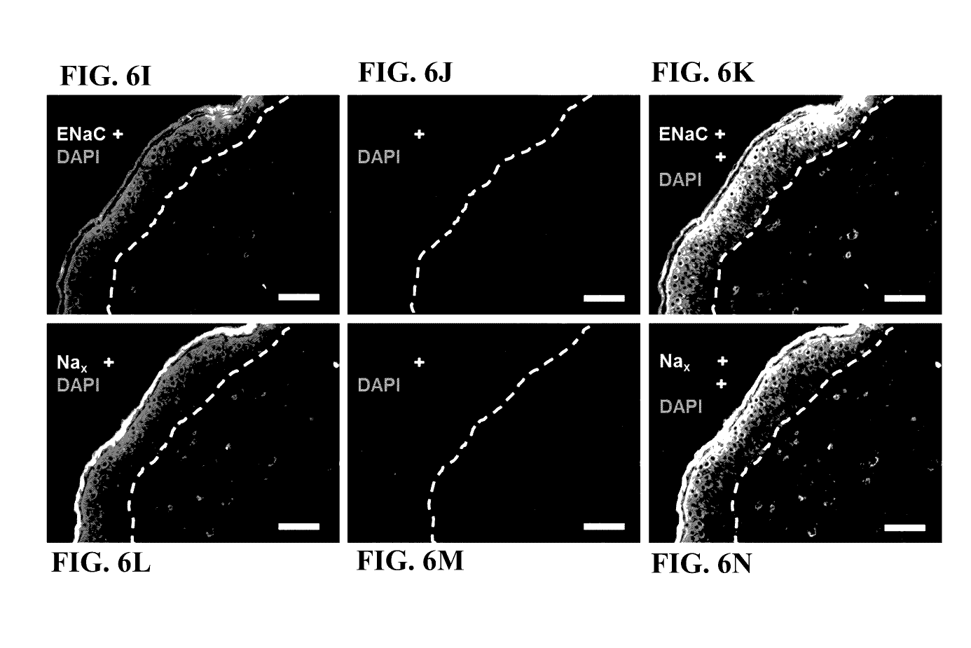

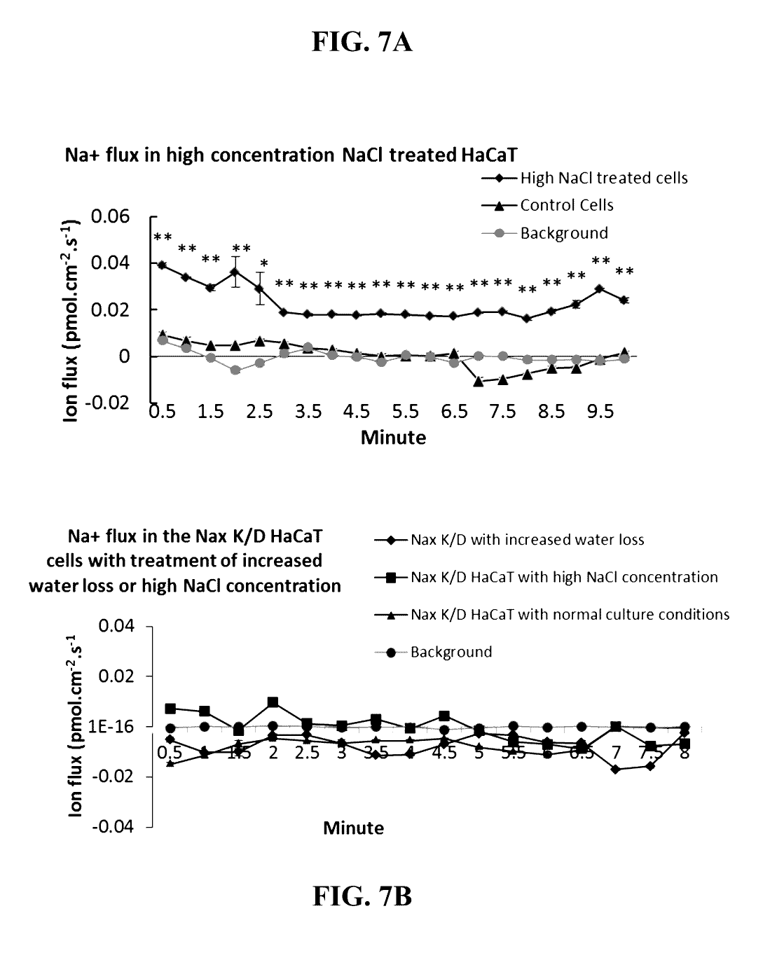

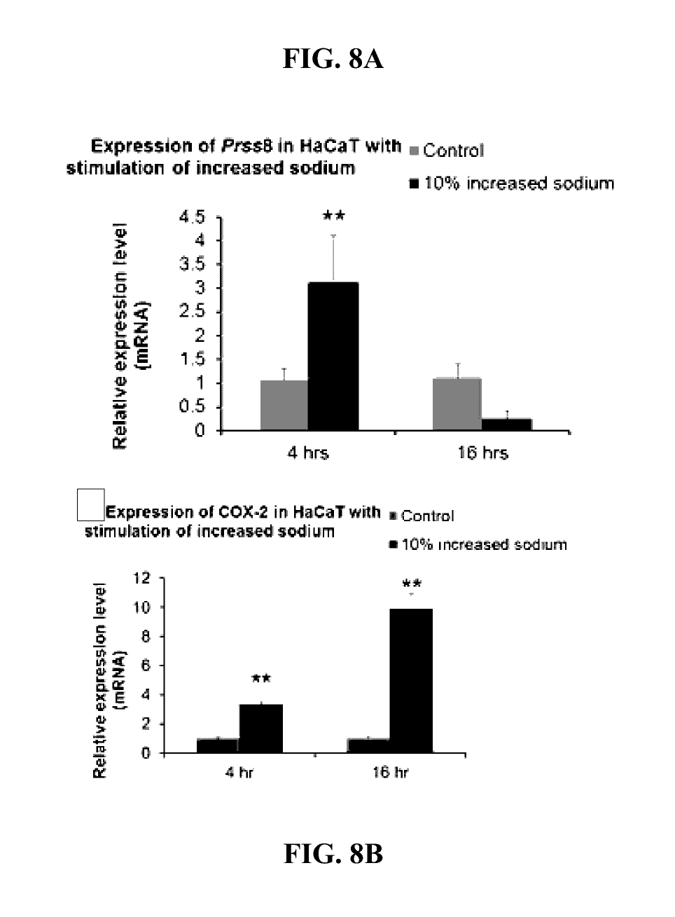

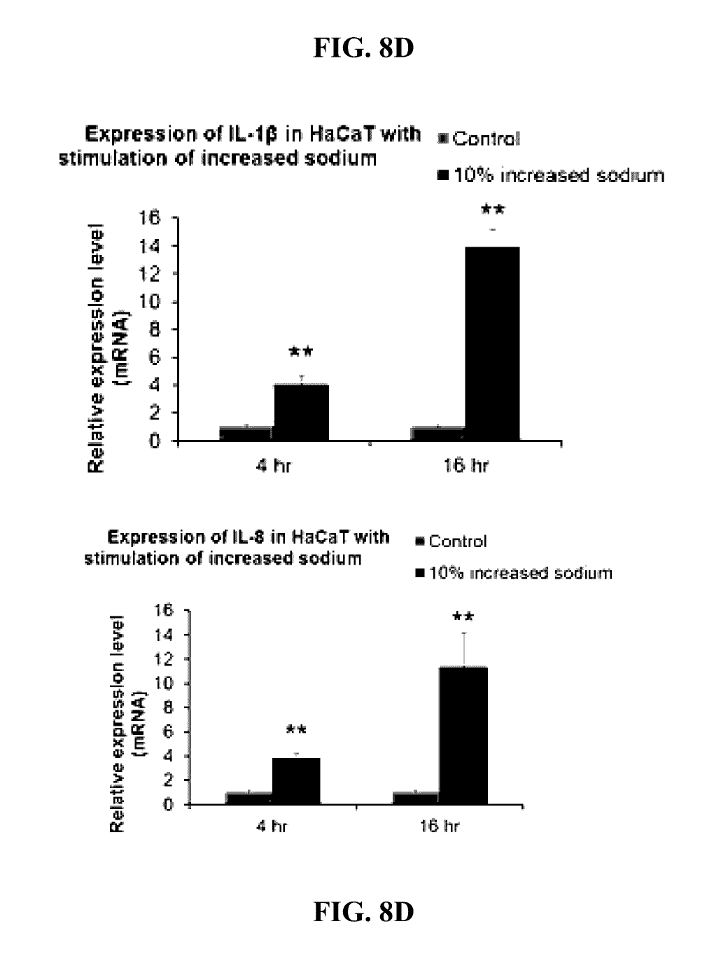

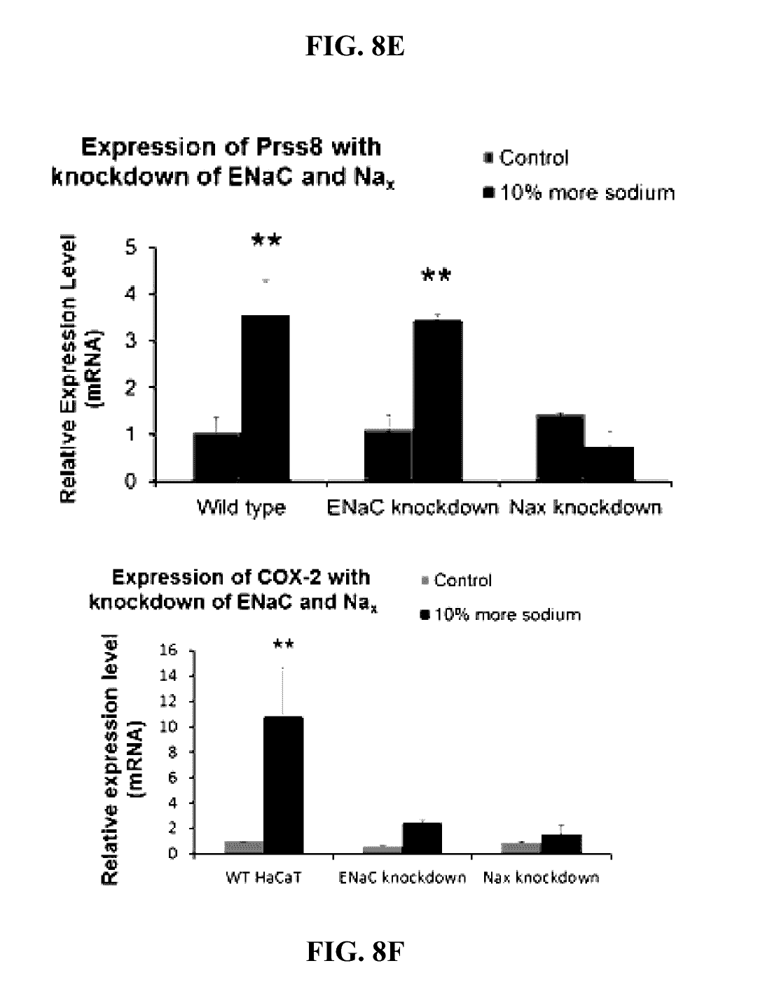

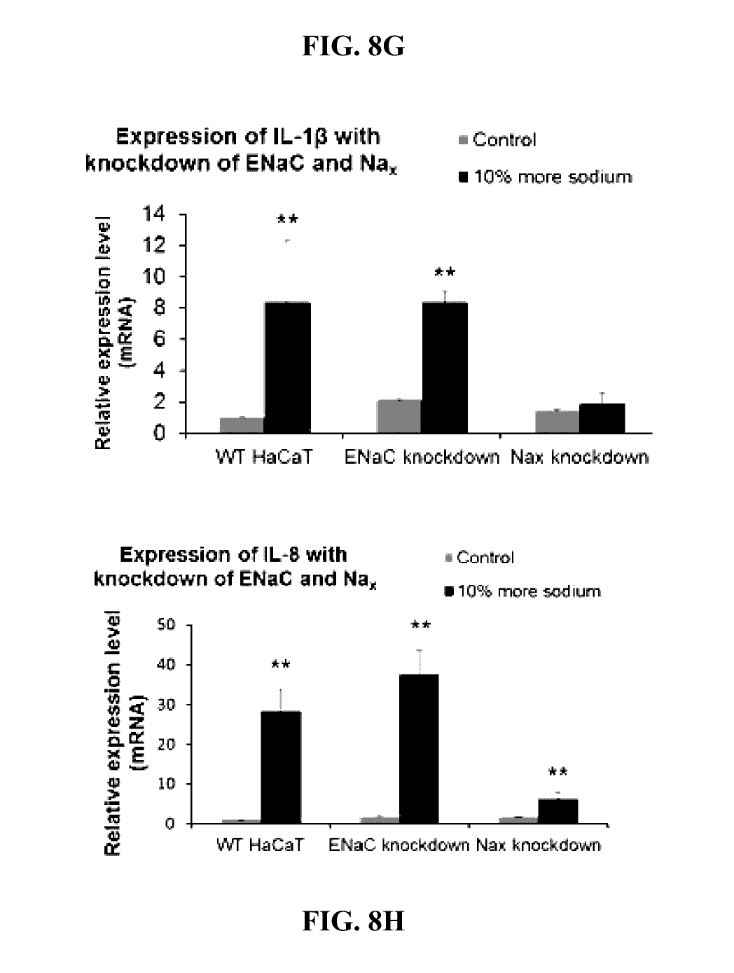

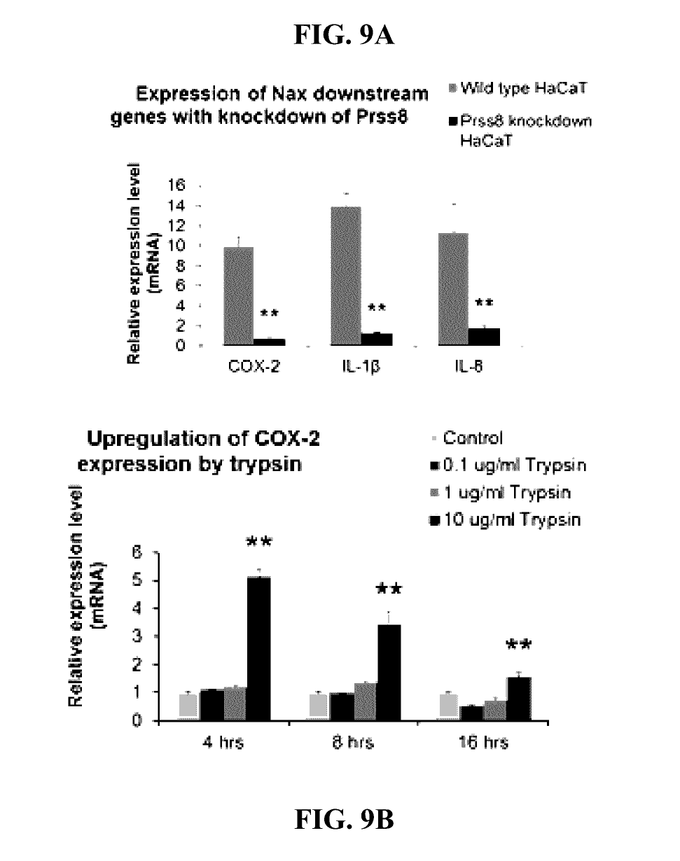

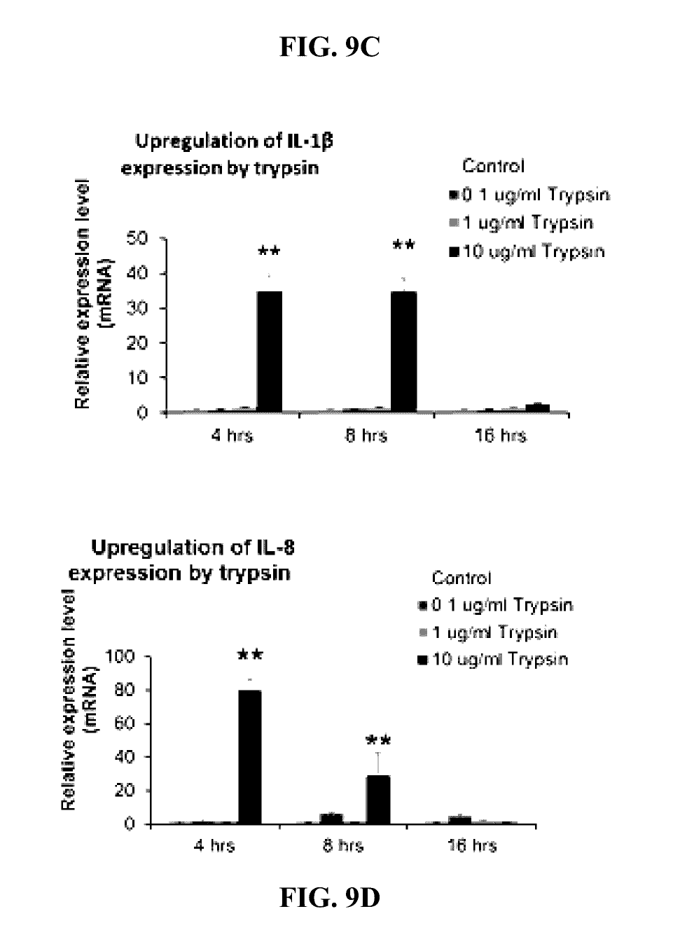

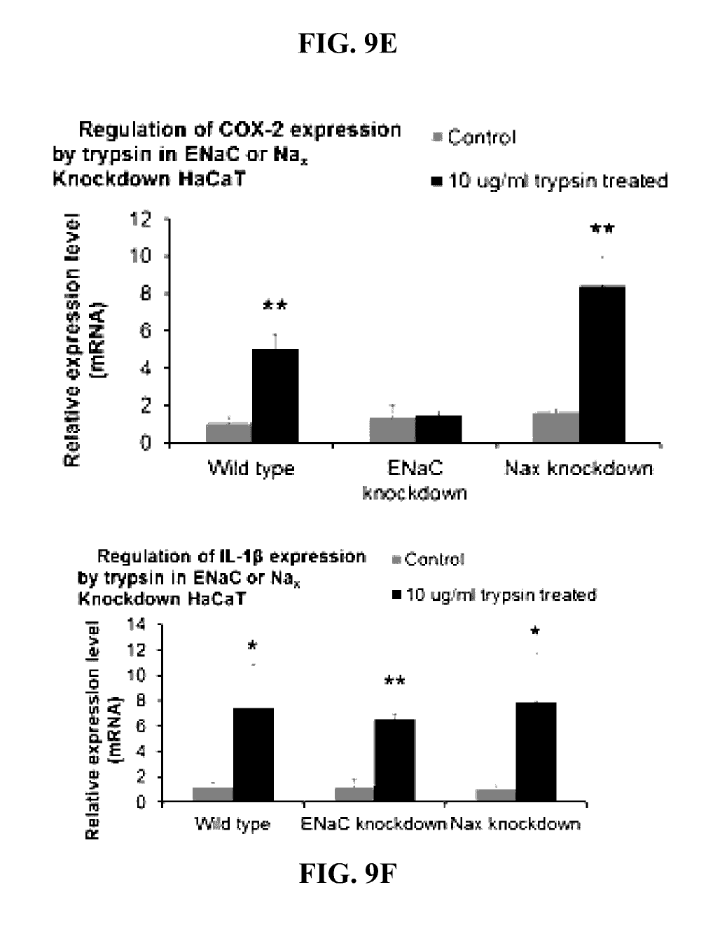

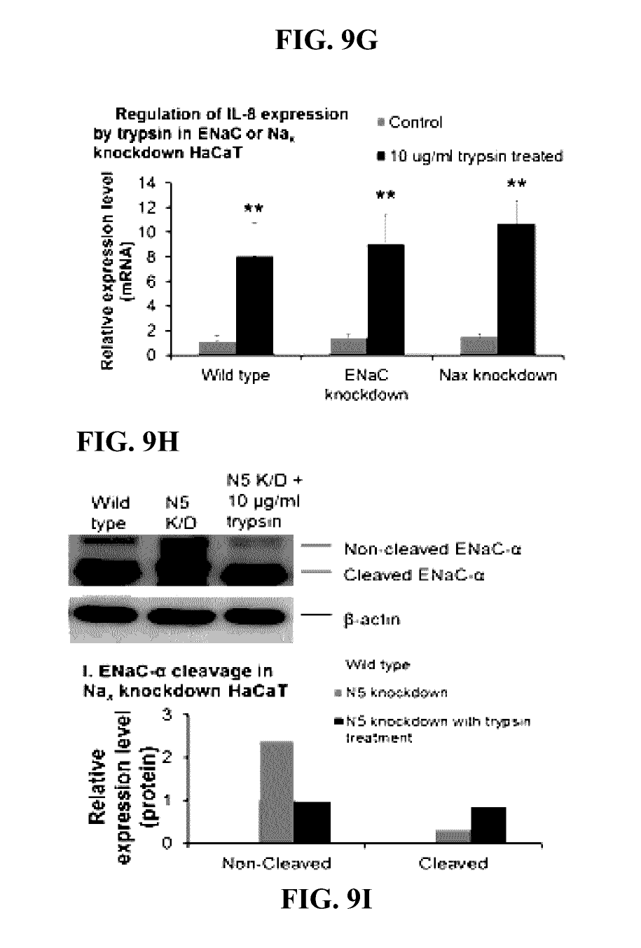

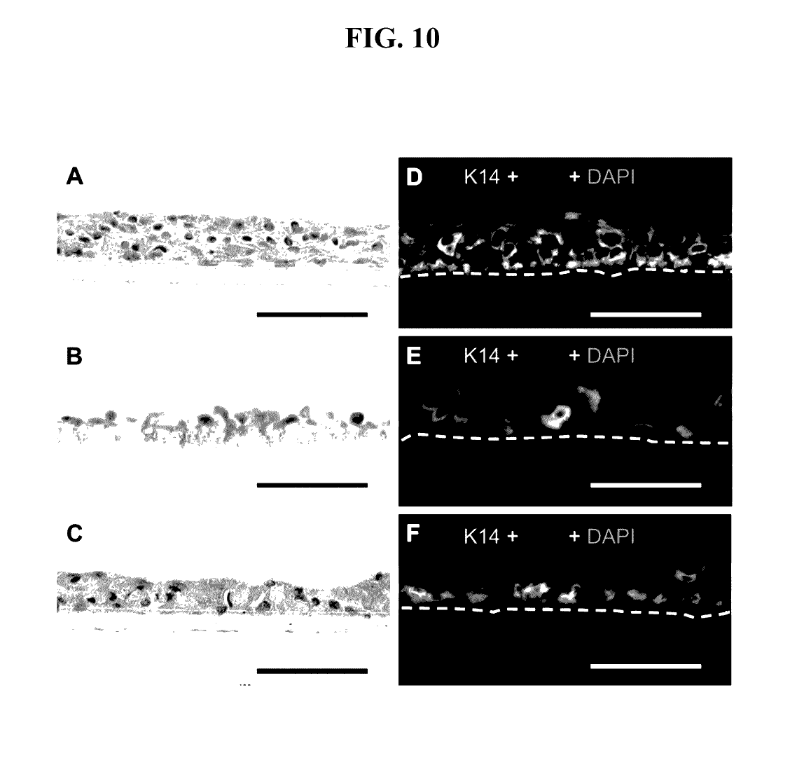

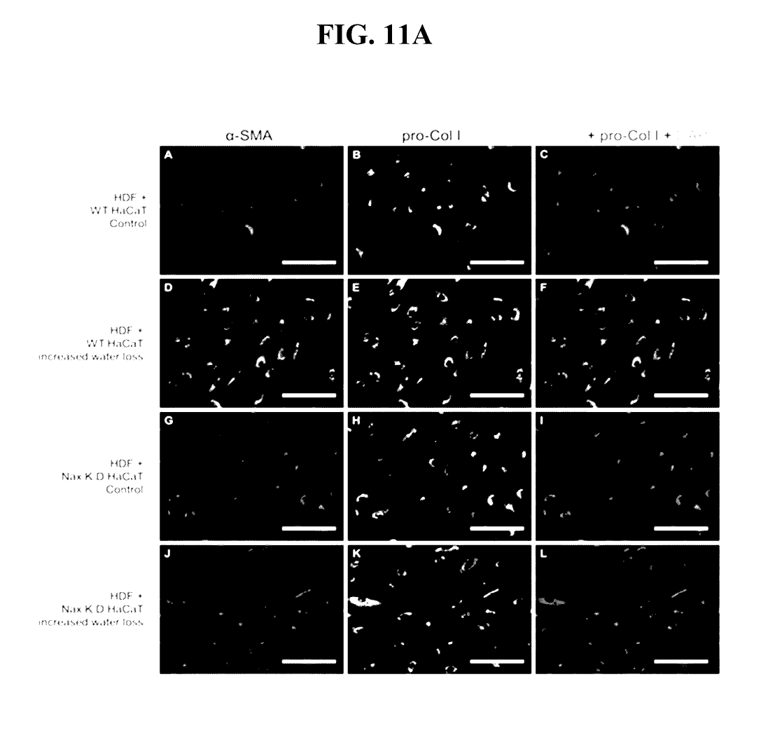

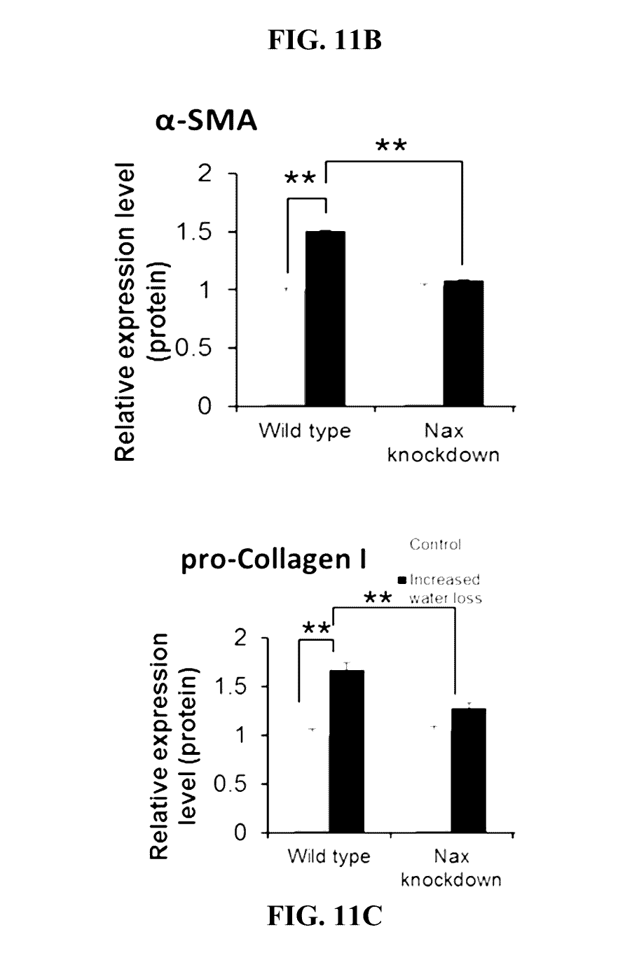

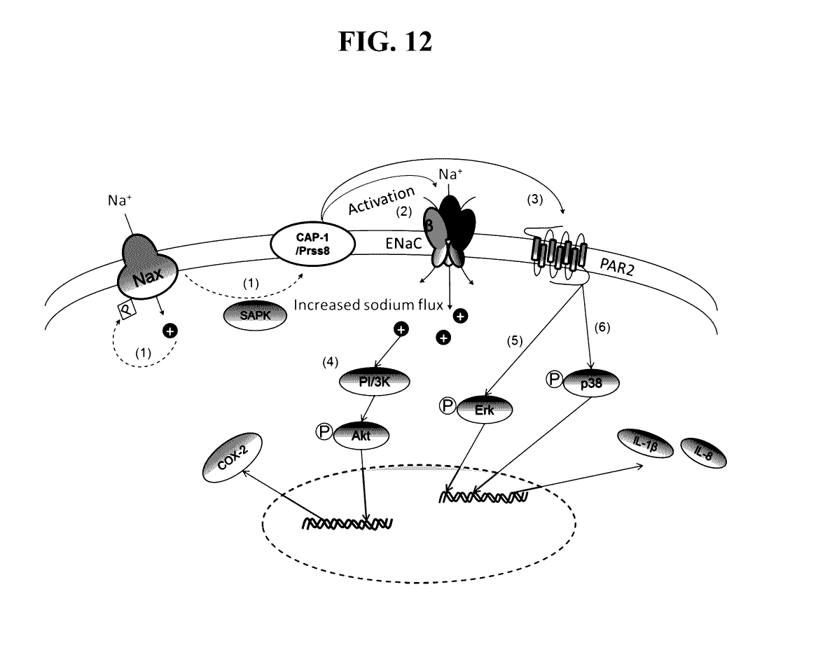

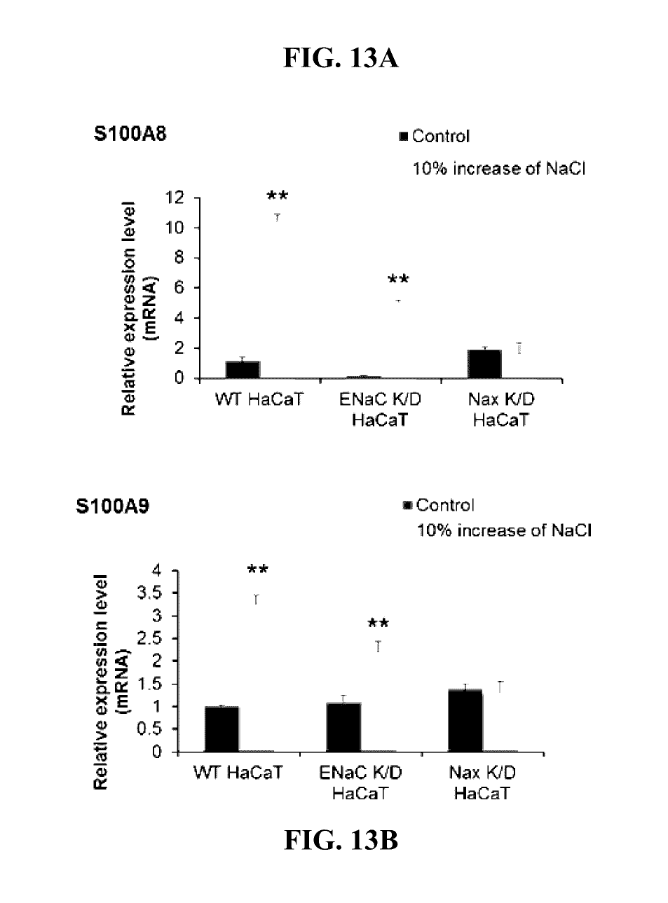

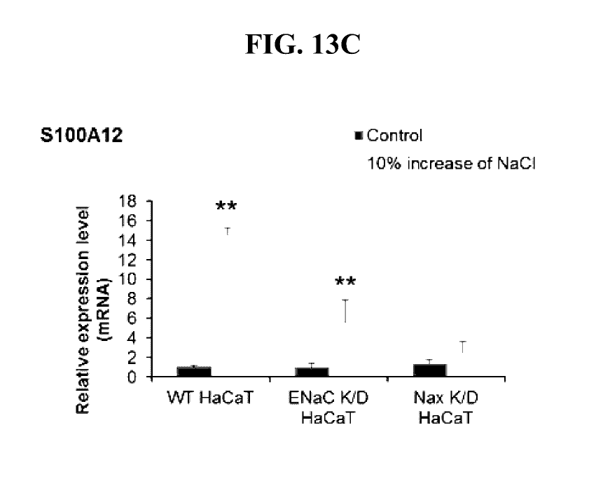

Sustained activation of dermal fibroblasts with accumulation of collagen is the main cause of hypertrophic scars (38, 39). The in vitro data above demonstrated that the ENaC-COX2 pathway is critical for the regulation of dermal fibroblast activation by epidermal keratinocytes. The role of the ENaC-COX-2 pathway was investigated in vivo utilizing a well validated hypertrophic scar model in the rabbit ear (31, 40-43). Amiloride, an ENaC antagonist, was topically applied to the rabbit ear wounds after re-epithelialization was complete. Control scars, located on the contralateral ear, were treated with vehicle alone. In this model, absence of scar elevation gives a scar elevation index (SEI) of 1.0, and control wounds heal with an elevated scar (SEI>1). Remarkably, rabbit ear wounds treated with 0.5 mg/wound of amiloride dramatically decreased the SEI from 1.42±0.08 to 1.06±0.05 (FIGS. 5A, B, and F). This reduction in scarring exceeds other therapies in validated model of hypertrophic scarring (14, 44, 45). In an identically designed experiment, Celecoxib, a COX-2 inhibitor, was applied on rabbit ear wounds to evaluate its effect on formation of hypertrophic scars. Similarly, treatment of 2 mg/wound of Celecoxib on rabbit ear wounds reduced the hypertrophy of the scar from 1.53±0.10 to 1.23±0.07 (FIGS. 5C, D, and G). However, lower concentrations of Amiloride or Celecoxib did not show significant reduction of scar formation compared to control wound healing groups (FIGS. 5F and 5G).

The epidermis plays a critical barrier function which maintains the hydration level of the deeper layers of skin. Increased TEWL occurs immediately upon injury to the lipid containing stratum corneum. Water loss of the skin causes alteration of expression levels of many genes involved in skin barrier lipid synthesis (46), keratinocyte differentiation and desquamation (47). However, the mechanism of gene regulation in response to TEWL remains largely unknown due to the lack of methods to detect the change of the microenvironment caused by TEWL in situ. It was hypothesized that increased TEWL resulted in increased sodium flux, which was confirmed with the use the SIET which demonstrated a high sensitivity to detect the ion flux under 0.01 pmol·cm-2·s-1. To reduce background to acceptable levels, cells were first treated with conditions of increased TEWL or sodium concentration, and then transferred to buffer with low sodium concentration and then measuring sodium flux compared to controls. Both keratinocytes and HESC showed increased similar sodium fluxes to increased TEWL (exposure to air with water evaporation) or 10% increased sodium, a level reached in the extracellular fluid in a water deprived thirsty animal (48).

Developed by Shipley and Feijo in 1999 (49), the SIET is an excellent tool to measure ion fluxes in vivo since the microelectrode can be non-invasively manipulated very close around to live cells or tissues. The measurement of SIET on HESCs and keratinocytes reliably reflected the sodium consumption during the water loss. Interestingly, the sodium flux level in HESCs was measured to much higher than the differentiated keratinocyte cultures. This may reflect the difference between this two skin substitutes. The HESC model is human skin grown in vitro. It contains all the layers of differentiated keratinocytes, including stratum basale, stratum spinosum, stratum granulosum, and stratum lucidum (the stratum corneum was removed before use by tape stripping). Previous studies have demonstrated that ENaC is preferentially expressed in differentiated keratinocytes (50). Since the cultured keratinocytes in this Example were only grown into 3-4 layers, they may have not been as differentiated as HESCs and may have expressed lower levels of ENaC. This may account for the stronger sodium fluxes detected on HESCs compared to keratinocytes. In addition the HESCs have a complex organization which may have other effects on sodium flux. Nevertheless, both models showed same trend of sodium flux in response to the TEWL and high sodium concentration.

Prior to this invention, the primary signal cascade, which is activated in response to TEWL, was believed to be unknown. Based on the results obtained from this Example, increased epithelial water loss raises extracellular sodium concentration which activates ENaC resulting in an influx of sodium into keratinocytes. Recently, ENaC has been demonstrated to play a critical role in embryo implantation in the uterus through increased COX-2 expression by endometrial epithelial cells. In this system, calcium was found to be the secondary messenger to initiate the phosphorylation of cAMP response element-binding protein (CREB) in response to sodium influx (51). In this Example, SIET did not detect the appearance of calcium influx upon the stress of increased water loss on keratinocytes. To further confirm this, the calcium channel was blocked by using benidipine hydrochloride (52).

The qPCR with the calcium blocked keratinocytes suggested that the increased expression levels of COX-2 in HKs during increased water loss or sodium concentration were not abolished by blocking the calcium channel, although there was slight decrease in mRNA. Since the calcium dependent pathway is not the only way to activate CREB as a transcription factor of COX-2, the ENaC may follow another pathway to phosphorylate the CREB in skin keratinocytes. Previous studies suggested that CREB phosphorylation can be performed through several routes, such as Grb2/ERK1 in retina (53), PI3k/Akt in kidney (54) and pancreatic ductal epithelial cells (55), and PKA/p38/MSK1 in fibroblasts (56). The western results generated during this Example suggested that the phosphorylation of Akt were rapidly increased as early as 10 min post stimulation (FIG. 2D). Meanwhile, the inhibition of PI3K also led to a remarkably drop of COX-2 expression level by increased sodium flux. Taken together, the Akt/PI3K is likely to be the pathway employed by ENaC to activate COX-2 expression in skin keratinocytes. However, whether the PI3K can directly sense the sodium influx upon stimulation of ENaC or there are some other components as intermediary agent between them is not necessary to know or understand to practice the present invention.

Taken that the ENaC activation in keratinocytes plays an important role in activation of dermal fibroblasts, antagonists targeting ENaC are useful for preventing or reducing scarring, as well as other skin conditions with increased TEWL. The ability to treat locally would limit toxicity concerns. The remarkable success of the antagonist used in this Example, amiloride, is a commercially available component that has been used in the treatment of hypertension (57) and congestive heart failure (58). The study of amiloride in rabbit ear scar treatment clearly showed the improvement of the scar formation at 28 days post wounding. Similarly, the inhibition of COX-2 activity with Celecoxib also contributed to the reduction of hypertrophy of rabbit ear scar.

While the present invention is not limited to any mechanism, it is believed that the increased water loss upon disruption of skin barrier results in a change in sodium concentration, which is sensed by ENaC. The intake of sodium in response to the activation of ENaC results in the phosphorylation of Akt through PI3K, which leads to the increase of COX-2 synthesis. The regulation of COX-2 at the transcription level is not clear although recent studies on the critical steps for embryo implantation in the uterus suggests that the CREB is the factor that initiates the production of COX-2 in epithelial cells of uterus (51). The up-regulation of COX-2 promotes the production and release of PGE2 to the matrix, which dramatically stimulates the dermal fibroblasts through receptor EP2 and EP4 to contribute to the hypertrophic scar formation (FIG. 5H). This indicates that molecules involved in ENaC-COX2 pathway may be used as pharmaceutical targets against skin hypertrophic scar.

### REFERENCES

1. Mustoe T A (2004) Scars and keloids. BMJ (Clinical research ed 328(7452):1329-1330.
2. Rhett J M, et al. (2008) Novel therapies for scar reduction and regenerative healing of skin wounds. Trends in biotechnology 26(4):173-180.
3. Miller M C & Nanchahal J (2005) Advances in the modulation of cutaneous wound healing and scarring. BioDrugs 19(6):363-381.
4. Kose O & Waseem A (2008) Keloids and hypertrophic scars: are they two different sides of the same coin? Dermatol Surg 34(3):336-346.
5. Suetake T, Sasai S, Zhen Y X, Ohi T, & Tagami H (1996) Functional analyses of the stratum corneum in scars. Sequential studies after injury and comparison among keloids, hypertrophic scars, and atrophic scars. Archives of dermatology 132(12):1453-1458.
6. Elias P M (2005) Stratum corneum defensive functions: an integrated view. The Journal of investigative dermatology 125(2):183-200.
7. Mustoe T A (2008) Evolution of silicone therapy and mechanism of action in scar management. Aesthetic plastic surgery 32(1):82-92.
8. Loden M (2003) Role of topical emollients and moisturizers in the treatment of dry skin barrier disorders. American journal of clinical dermatology 4(11):771-788.
9. Szpaderska A M, Zuckerman J D, & DiPietro L A (2003) Differential injury responses in oral mucosal and cutaneous wounds. Journal of dental research 82(8):621-626.
10. Mak K, et al. (2009) Scarless healing of oral mucosa is characterized by faster resolution of inflammation and control of myofibroblast action compared to skin wounds in the red Duroc pig model. Journal of dermatological science 56(3):168-180.
11. Ahn S T, Monafo W W, & Mustoe T A (1989) Topical silicone gel: a new treatment for hypertrophic scars. Surgery 106(4):781-786; discussion 786-787.
12. Ahn S T, Monafo W W, & Mustoe T A (1991) Topical silicone gel for the prevention and treatment of hypertrophic scar. Arch Surg 126(4):499-504.
13. Mustoe T A, et al. (2002) International clinical recommendations on scar management. Plast Reconstr Surg 110(2):560-571.
14. Saulis A S, Chao J D, Telser A, Mogford J E, & Mustoe T A (2002) Silicone occlusive treatment of hypertrophic scar in the rabbit model. Aesthet Surg J 22(2):147-153.
15. De Jongh C M, et al. (2006) Stratum corneum cytokines and skin irritation response to sodium lauryl sulfate. Contact Dermatitis 54(6):325-333.
16. Sogabe Y, et al. (2002) Functions of the stratum corneum in systemic sclerosis as distinct from hypertrophic scar and keloid functions. Journal of dermatological science 29(1):49-53.
17. Lee S H, et al. (1992) Calcium and potassium are important regulators of barrier homeostasis in murine epidermis. J Clin Invest 89(2):530-538.
18. Brouard M, Casado M, Djelidi S, Barrandon Y, & Farman N (1999) Epithelial sodium channel in human epidermal keratinocytes: expression of its subunits and relation to sodium transport and differentiation. Journal of cell science 112 (Pt 19):3343-3352.
19. Mauro T, et al. (2002) The ENaC channel is required for normal epidermal differentiation. The Journal of investigative dermatology 118(4):589-594.
20. Mobasheri A, Barrett-Jolley R, Shakibaei M, Canessa C M, & Martin-Vasallo P (2005) Enigmatic Roles of the Epithelial Sodium Channel (ENaC) in Articular Chondrocytes and Osteoblasts: Mechanotransduction, Sodium Transport or Extracellular Sodium Sensing?
21. Loffing J & Schild L (2005) Functional domains of the epithelial sodium channel. Journal of the American Society of Nephrology: JASN 16(11):3175-3181.
22. Chalfant M L, et al. (1999) Intracellular H+ regulates the alpha-subunit of ENaC, the epithelial Na+ channel. The American journal of physiology 276(2 Pt 1):C477-486.
23. Masilamani S, Kim G H, Mitchell C, Wade J B, & Knepper M A (1999) Aldosterone-mediated regulation of ENaC alpha, beta, and gamma subunit proteins in rat kidney. J Clin Invest 104(7):R19-23.
24. Titze J, et al. (2004) Glycosaminoglycan polymerization may enable osmotically inactive Na+ storage in the skin. Am J Physiol Heart Circ Physiol 287(1):H203-208.
25. Rajnicek A M, Stump R F, & Robinson K R (1988) An endogenous sodium current may mediate wound healing in Xenopus neurulae. Dev Biol 128(2):290-299.
26. Habibipour S, et al. (2003) Effect of sodium diphenylhydantoin on skin wound healing in rats. Plast Reconstr Surg 112(6):1620-1627.
27. Matsuzaki T, Suzuki T, Koyama H, Tanaka S, & Takata K (1999) Water channel protein AQP3 is present in epithelia exposed to the environment of possible water loss. J Histochem Cytochem 47(10):1275-1286.
28. Lee Y, et al. (2012) Changes in transepidermal water loss and skin hydration according to expression of aquaporin-3 in psoriasis. Ann Dermatol 24(2):168-174.
29. Hamm L L, Feng Z, & Hering-Smith K S (2010) Regulation of sodium transport by ENaC in the kidney. Curr Opin Nephrol Hypertens 19(1):98-105.
30. Deng W, Li C Y, Tong J, Zhang W, & Wang D X (2012) Regulation of ENaC-mediated alveolar fluid clearance by insulin via PI3K/Akt pathway in LPS-induced acute lung injury. Respir Res 13:29.
31. Gallant-Behm C L, et al. (2011) Epithelial regulation of mesenchymal tissue behavior. The Journal of investigative dermatology 131(4):892-899.
32. Buj old K, et al. (2009) CD36-mediated cholesterol efflux is associated with PPARgamma activation via a MAPK-dependent COX-2 pathway in macrophages. Cardiovasc Res 83(3):457-464.
33. Singer C A, et al. (2003) p38 MAPK and NF-kappaB mediate COX-2 expression in human airway myocytes. Am J Physiol Lung Cell Mol Physiol 285(5):L1087-1098.
34. Xia S, Zhao Y, Yu S, & Zhang M (2010) Activated PI3K/Akt/COX-2 pathway induces resistance to radiation in human cervical cancer HeLa cells. Cancer Biother Radiopharm 25(3):317-323.
35. Zhang K, Rekhter M D, Gordon D, & Phan S H (1994) Myofibroblasts and their role in lung collagen gene expression during pulmonary fibrosis. A combined immunohistochemical and in situ hybridization study. Am J Pathol 145(1):114-125.
36. Broekema M, et al. (2007) Bone marrow-derived myofibroblasts contribute to the renal interstitial myofibroblast population and produce procollagen I after ischemia/reperfusion in rats. Journal of the American Society of Nephrology: JASN 18(1):165-175.
37. Sandulache V C, et al. (2009) Prostaglandin E2 is activated by airway injury and regulates fibroblast cytoskeletal dynamics. Laryngoscope 119(7):1365-1373.
38. Wang J, Dodd C, Shankowsky H A, Scott P G, & Tredget E E (2008) Deep dermal fibroblasts contribute to hypertrophic scarring. Lab Invest 88(12):1278-1290.
39. Wang J, et al. (2011) Toll-like receptors expressed by dermal fibroblasts contribute to hypertrophic scarring. J Cell Physiol 226(5):1265-1273.
40. Ko J H, Kim P S, Zhao Y, Hong S J, & Mustoe T A (2012) HMG-CoA reductase inhibitors (statins) reduce hypertrophic scar formation in a rabbit ear wounding model. Plast Reconstr Surg 129(2):252e-261e.
41. Gallant-Behm C L & Mustoe T A (2010) Occlusion regulates epidermal cytokine production and inhibits scar formation. Wound Repair Regen 18(2):235-244.
42. O'Shaughnessy K D, De La Garza M, Roy N K, & Mustoe T A (2009) Homeostasis of the epidermal barrier layer: a theory of how occlusion reduces hypertrophic scarring. Wound Repair Regen 17(5):700-708.
43. Sisco M, et al. (2008) Antisense inhibition of connective tissue growth factor (CTGF/CCN2) mRNA limits hypertrophic scarring without affecting wound healing in vivo. Wound Repair Regen 16(5):661-673.
44. Mustoe T A, et al. (1987) Accelerated healing of incisional wounds in rats induced by transforming growth factor-beta. Science 237(4820):1333-1336.
45. Morris D E, et al. (1997) Acute and chronic animal models for excessive dermal scarring: quantitative studies. Plast Reconstr Surg 100(3):674-681.
46. Buraczewska I, Berne B, Lindberg M, Loden M, & Torma H (2009) Moisturizers change the mRNA expression of enzymes synthesizing skin barrier lipids. Arch Dermatol Res 301(8):587-594.
47. Buraczewska I, Berne B, Lindberg M, Loden M, & Torma H (2009) Long-term treatment with moisturizers affects the mRNA levels of genes involved in keratinocyte differentiation and desquamation. Arch Dermatol Res 301(2):175-181.
48. Hiyama T Y, et al. (2002) Na(x) channel involved in CNS sodium-level sensing. Nature neuroscience 5(6):511-512.
49. Shipley A M & Feijó J A (1999) The use of the vibrating probe technique to study steady extracellular currents during pollen germination and tube growth. Fertilization in Higher Plants: Molecular and Cytological Aspects, eds Cresti M, Cai G, & Moscatelli S (Springer-Verlag. Heidelberg, Berlin), pp 235-252.
50. Guitard M, Leyvraz C, & Hummler E (2004) A nonconventional look at ionic fluxes in the skin: lessons from genetically modified mice. News Physiol Sci 19:75-79.
51. Ruan Y C, et al. (2012) Activation of the epithelial Na(+) channel triggers prostaglandin E(2) release and production required for embryo implantation. Nat Med.
52. Seino H, et al. (2007) Effect of benidipine hydrochloride, a long-acting T-type calcium channel blocker, on blood pressure and renal function in hypertensive patients with diabetes mellitus. Analysis after switching from cilnidipine to benidipine. Arzneimittelforschung 57(8):526-531.
53. Racz B, et al. (2006) Involvement of ERK and CREB signaling pathways in the protective effect of PACAP in monosodium glutamate-induced retinal lesion. Ann N Y Acad Sci 1070:507-511.
54. Du K & Montminy M (1998) CREB is a regulatory target for the protein kinase Akt/PKB. J Biol Chem 273(49):32377-32379.
55. Li X Y, Zhan X R, Liu X M, & Wang X C (2011) CREB is a regulatory target for the protein kinase Akt/PKB in the differentiation of pancreatic ductal cells into islet beta-cells mediated by hepatocyte growth factor. Biochem Biophys Res Commun 404(2):711-716.
56. Delghandi M P, Johannessen M, & Moens U (2005) The cAMP signalling pathway activates CREB through PKA, p38 and MSK1 in NIH 3T3 cells. Cell Signal 17(11):1343-1351.
57. Baker E H, et al. (2002) Amiloride, a specific drug for hypertension in black people with T594M variant? Hypertension 40(1):13-17.
58. Cheitlin M D, Byrd R, Benowitz N, Liu E, & Modin G (1991) Amiloride improves hemodynamics in patients with chronic congestive heart failure treated with chronic digoxin and diuretics. Cardiovasc Drugs Ther 5(4):719-725.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating a skin condition comprising:
applying a composition to an affected skin surface of a subject with dermatitis, psoriasis, or a cutaneous wound,
wherein said composition comprises a therapeutic amount of a therapeutic agent, and wherein said therapeutic agent is an inhibitor of Naₓ (SCN7A) mRNA or protein.

2. The method of claim 1, wherein said composition further comprises a topical component suitable for application to said affected skin surface.

3. The method of claim 1, wherein said inhibitor of Naₓ/SCN7A mRNA or protein is siRNA or miRNA directed to Naₓ/SCN7A.

4. The method of claim 1, wherein said inhibitor of Naₓ (SCN7A) mRNA or protein is an antibody which is an inhibitor of Naₓ/SCN7A protein.
